(19) United States
(12) Patent Application Publication
(10) Pub. No.: US 2011/0059189 A1
(43) Pub. Date: Mar. 10, 2011

(54) METHOD AND COMPOSITION FOR TREATING CANCER, EFFECTING APOPTOSIS AND TREATING RETROVIRAL INFECTIONS

(75) Inventor: Ignacio CISNEROS, Odessa, TX (US)

(73) Assignee: CISNE ENTERPRISES INC., Odessa, TX (US)

(21) Appl. No.: 12/845,220

(22) Filed: Jul. 28, 2010

Related U.S. Application Data
(60) Provisional application No. 61/240,423, filed on Sep. 8, 2009.

(51) Int. Cl. A61K 33/00; A61P 31/12; A61P 35/00

(52) U.S. Cl. 424/722

(57) ABSTRACT

A modified sodium silicate composition, and methods of treating cancer and viral infections utilizing the modified sodium silicate composition (Na_{8.2}Si_{4.4}H_{9.7}O_{17.6}). Na_{8.2}Si_{4.4}H_{9.7}O_{17.6} can be administered to increase the nitric oxide concentration in the body, effect apoptosis, increase NO formation by neutrophils. Inhibit cell mutations, and inhibit oxidative stress.

### CROSS-REFERENCE TO RELATED APPLICATIONS

The instant application claims priority from provisional Application No. 61/240,423, filed Sep. 8, 2009, the entire contents of each of which are hereby incorporated by reference.

### FIELD OF INVENTION

The application relates to a silicon-based alkaline composition, methods and compositions for treating cancer and retroviral infections such as HIV infection, effecting apoptosis, increasing NO formation by neutrophils, inhibiting cell mutations, inhibiting oxidative stress.

### BACKGROUND OF THE INVENTION

Cancer is characterized by the proliferation of cells that are not subject to normal cell proliferating controls. It is a major cause of death in humans and other mammals. Uncontrolled proliferation is a trait of cancer cells. Cancer is treated by radiation therapy, chemotherapy, surgery, hyperthermia, laser, photodynamic therapy, inhibition of angiogenesis, bone marrow transplantation, and gene therapy.

Unfortunately, cancer cells often become resistant to standard therapies, and the cancer cells, rather than undergoing apoptosis after several divisions, resist the chemotherapy and continue to multiply.

Acquired immunodeficiency syndrome (AIDS), caused by human immunodeficiency virus (HIV), is an immunosuppressive disease that results in life-threatening opportunistic infections and malignancies. Despite continuous advances made in anti-retroviral therapy, AIDS has become the leading cause of death in Africa and fourth worldwide; the number of people with HIV is increasing at an alarming rate in India and Southeast Asia. Two major types of HIV have been identified so far, HIV-1 and HIV-2. HIV-1 is the cause of the worldwide epidemic and is most commonly referred to as HIV. It is a highly variable virus, which mutates readily. Several therapeutic drugs have been developed to control the onset of AIDS in the carriers of this virus, such as reverse transcriptase inhibitors (ZDV and AZT) and protease inhibitors that suppress HIV replication. While the infections in developed countries have been suppressed with these drugs, there are several limitations which have prevented successful management of retro-viral diseases worldwide. These limitations include high cost and serious side effects such as inhibition of hematopoietic function and development of resistant strains of HIV.

Nitric oxide, NO, exerts anti-viral effects and inhibits viral replication. S-nitrosylation of thiol-proteases is a mechanism by which nitric oxide exerts anti-viral effects and inhibits viral replication. Additionally, nitric oxide is an important signaling molecule in hematopoeisis and myeloid differentiation. Increasing production of nitric oxide may alleviate some hematopoietic side effects of retroviral therapy.

Oxidative stress is caused by an imbalance between the production of reactive oxygen and a body's ability to readily detoxify the reactive intermediates or easily repair the resulting damage. In humans, oxidative stress is involved in many diseases, such as atherosclerosis, Parkinson's disease, heart failure, myocardial infarction, Alzheimer's disease, fragile X syndrome and chronic fatigue syndrome.

Oxidative stress results from an abnormal level of reactive oxygen species (ROS), which can occur as a result of fungal or viral infection, inflammation, aging, exposure to UV irradiation, pollution, excessive alcohol consumption and smoking.

Therefore, there is an urgent need to develop new and safer categories of therapeutic strategies that will manage these important public health concerns.

### SUMMARY OF THE INVENTION

The material that is the subject of the present application is a silicon-based alkaline composition of the formula of Na₈Si₄H_{9.7}O_{17.6} (hereinafter also referred to as modified sodium silicate). Modified sodium silicate is a modified value-added silicon-based compound. The present inventors have found that modified sodium silicate is effective in vitro against cancer cells and against the viral infection, in particular, infection caused by the HIV retrovirus. The present inventors further found that modified sodium silicate increases nitric oxide concentration in the body. Additionally, they found that modified sodium silicate has very high antimicrobial effect and can reduce the risk of infections and poisoning associated with several pathogens in both humans and animals. Moreover, the modified sodium silicate was found to reduce reactive oxygen, species, thus lowering oxidative stress.

Thus, the present application provides for a novel silicon-based alkaline composition, a pharmaceutical composition comprising the composition, and methods of treating various disease conditions by administering the composition.

In one embodiment, the present application provides a method for treating cancer in a subject in need thereof by causing one or more anti-cancer effects by administering to the patient an effective amount of the silicon-based alkaline composition as described herein, wherein the one or more anti-cancer effects are selected from the group consisting of preventing attachment of cancer cells; reducing harmful mutations in cellular DNA; inducing apoptosis; and stimulating anti-oxidant enzymes. In a preferred embodiment, the method of the present application provides for treating colon cancer.

In another embodiment, the present application provides for a method of preventing attachment of cancer cells; a method of reducing harmful mutations in cellular DNA; a method inducing apoptosis; and a method of stimulating anti-oxidant enzymes.

In another embodiment, the present application provides for a method for treating viral infection in a subject in need thereof by causing one or more anti-viral effects by administering to a patient an effective amount of the modified sodium silicate as described herein, wherein the one or more anti-viral effects are selected from the group consisting of increasing nitric oxide dependent anti-viral effects; inhibiting enzymes involved in viral assembly; causing changes in viral carbohydrate composition; and inhibiting viral enzymes responsible for transcribing RNA to DNA. In a preferred embodiment, the viral infection is a retroviral infection, for example, an infection caused by the human immunodeficiency virus (HIV).

In still another embodiment, the present application provides a method for treating oxidative stress in a patient in need thereof by administering to the patient an effective amount of the modified sodium silicate composition of the present invention. Reducing oxidative stress can be used to treat a variety of diseases, such as atherosclerosis, Parkinson's disease, heart failure, myocardial infarction, Alzheimer's disease, Fragile X, syndrome and chronic fatigue syndrome.

These and other features, aspects, and advantages of the subject matter of this application will become better understood with regard to the following description, appended claims, and accompanying drawings.

### BRIEF DESCRIPTION OF THE DRAWINGS

FIG. 1 is a schematic flow diagram of the process for making the composition.

FIG. 2 shows the effect of modified sodium silicate on the survival of colon cancer cell line HT-29.

FIG. 3 shows the effect of modified sodium silicate on attachment of colon cancer cell line HT-29 to surfaces.

FIG. 4 shows the effect of modified sodium silicate against various types of mutations induced by sodium azide in the Ames test for mutations.

FIG. 5 shows the apoptotic effect of modified sodium silicate at various concentrations as measured by fragmented DNA.

FIG. 6 shows the effect of modified sodium silicate at various concentrations on free radical formation.

FIG. 7 shows the effect of modified sodium silicate at various concentrations on SOD activity.

FIG. 8 shows the effect of v at various concentrations on catalase activity.

FIG. 9 shows the effect of modified sodium silicate at various concentrations on reduced glutathione levels.

FIG. 10 shows the effect of various concentrations of modified sodium silicate on nitric oxide levels as measured by total nitrates.

FIG. 11 shows the effect of various concentrations of modified sodium silicate on HIV envelope protein glucosylation.

FIG. 12 shows the effect of various concentrations of modified sodium silicate on HIV envelope protein glucuronylation.

FIG. 13 shows the effects of various concentrations of modified sodium silicate on ribose concentration.

FIG. 14 shows the effect of various concentrations of modified sodium silicate on heptose concentration.

FIG. 15 shows the effect of various concentrations of modified sodium silicate on sialic acid concentration.

FIG. 16 shows the effect of various concentrations of modified sodium silicate on uronic acid concentration.

FIG. 17 shows the effect of various concentrations of modified sodium silicate on HIV-1 reverse transcriptase activity.

FIG. 18 shows the effect of various concentrations of modified sodium silicate on HI-protease activity.

FIG. 19 shows the formula for trimeric sodium silicate (Na₂SiO₃)₃.

FIG. 20 shows the equilibrium formula for sodium silicate pentahydrate (Na₂SiO₃).5H₂O.

FIG. 21 is the FTIR spectrum of the modified sodium silicate product.

FIG. 22 is the ¹H MAS-NMR spectrum of the modified sodium silicate product.

FIG. 23 shows the effect of modified sodium silicate on SOD and catalase activity.

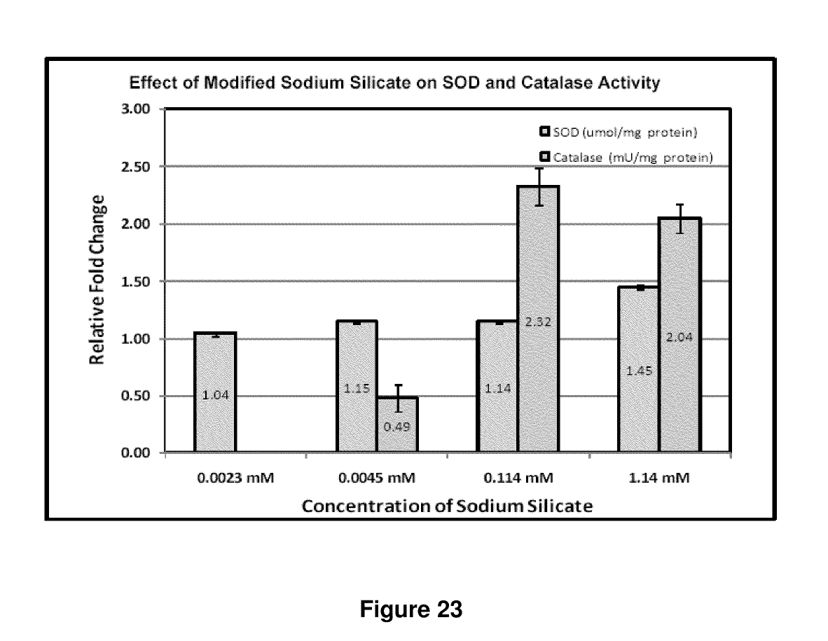

FIG. 24 shows the antimutagenic effect of modified sodium silicate based upon decrease in number of reversions.

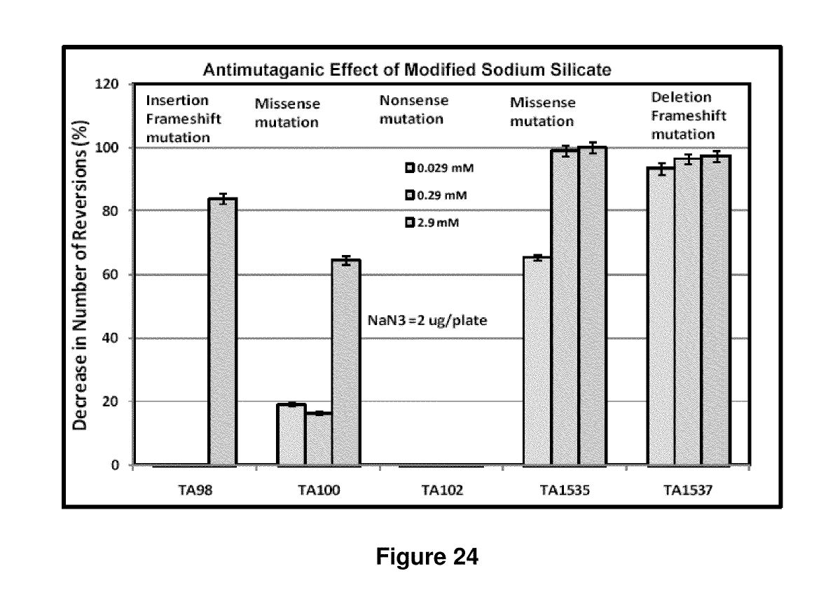

FIG. 25 effect of modified sodium silicate on GSH.

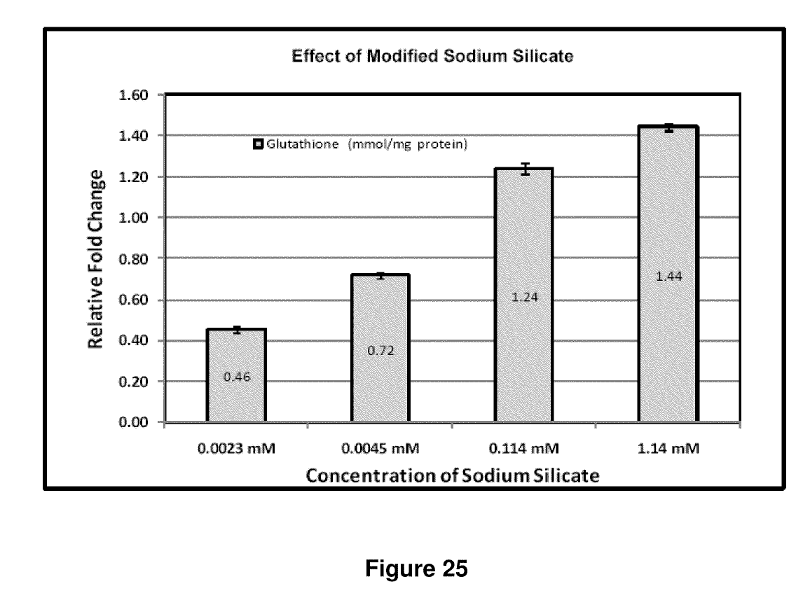

FIG. 26 shows the and the anti-proliferative and anti-adhesive effects of modified sodium silicate.

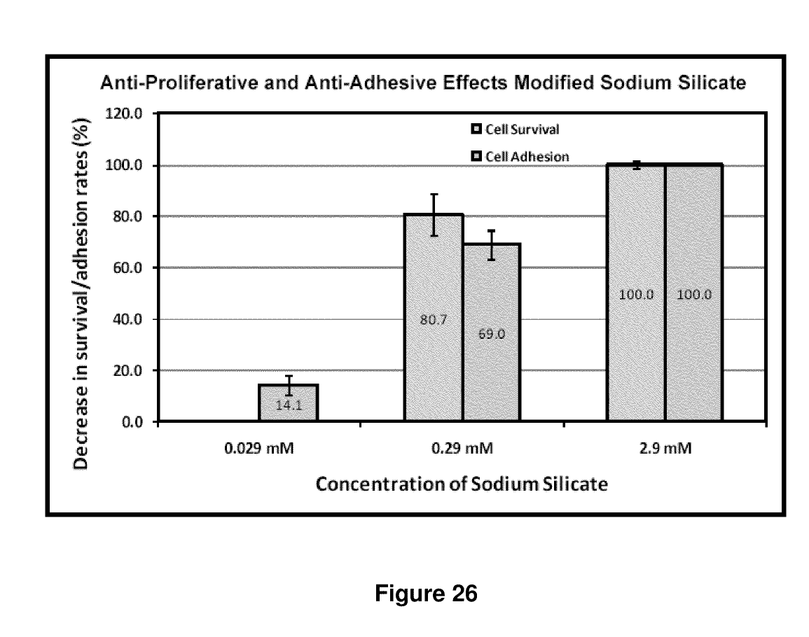

FIG. 27 shows the inhibition of protein glyucosylation by modified sodium silicate.

FIG. 28 shows inhibit of HIV-I reverse transcriptase by modified sodium silicate.

FIG. 29 shows the effect of modified sodium silicate on NO_{X}.

### DETAILED DESCRIPTION OF THE INVENTION

Having now generally described the subject matter of the application, the same will be more readily understood through reference to the following examples, which are provided by way of illustration and are not intended to be limiting of the subject matter of the application.

The material that is the subject of the present application is a silicon-based alkaline composition (also referred to herein as modified sodium silicate). MS and NMR analysis generated a putative empirical formula of the compound to be Na_{8.2}Si_{4.4}H_{9.7}O_{17.6}. The formula suggests that modified sodium silicate is not a single compound but a mixture of two different compounds that are in equilibrium with each other. Modified sodium silicate appears to be a mixture of:

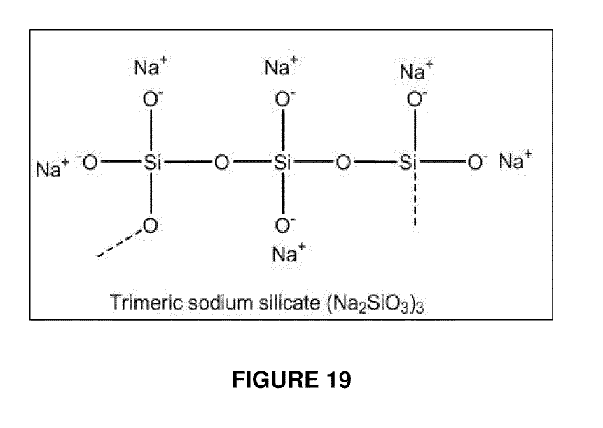

1. Trimeric Sodium Silicate (Na₂SiO₃)₃, shown in FIG. 19; and

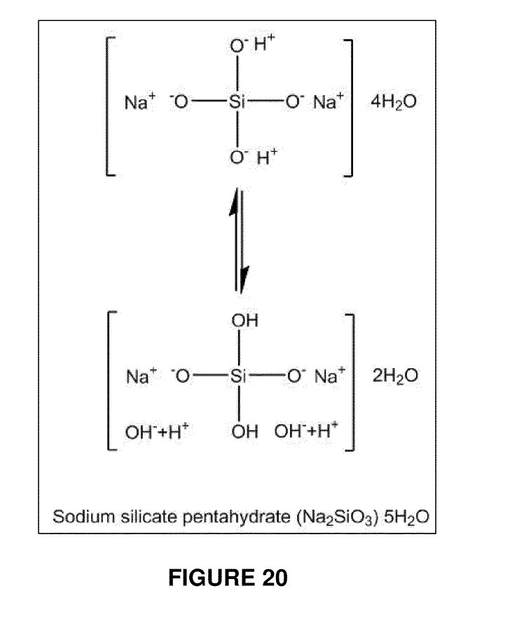

2. Sodium Silicate Pentahydrate (Na₂SiO₃).5H₂O, shown in FIG. 20.

Sodium Silicate Pentahydrate (Na₂SiO₃).5H₂O appears to exist in equilibrium with two structural forms (FIG. 20), with one form containing one ionized water molecule and the other form containing three ionized water molecules. Many of the biological activities of modified sodium silicate could be due to these multiple ionized forms, giving it the ability to accept and donate electrons and participate in important redox reactions in the body to bring about redox homeostasis.

The process for producing modified sodium silicate is described in copending provisional application No. 61/218,549, filed Jun. 19, 2009. This process is described below.

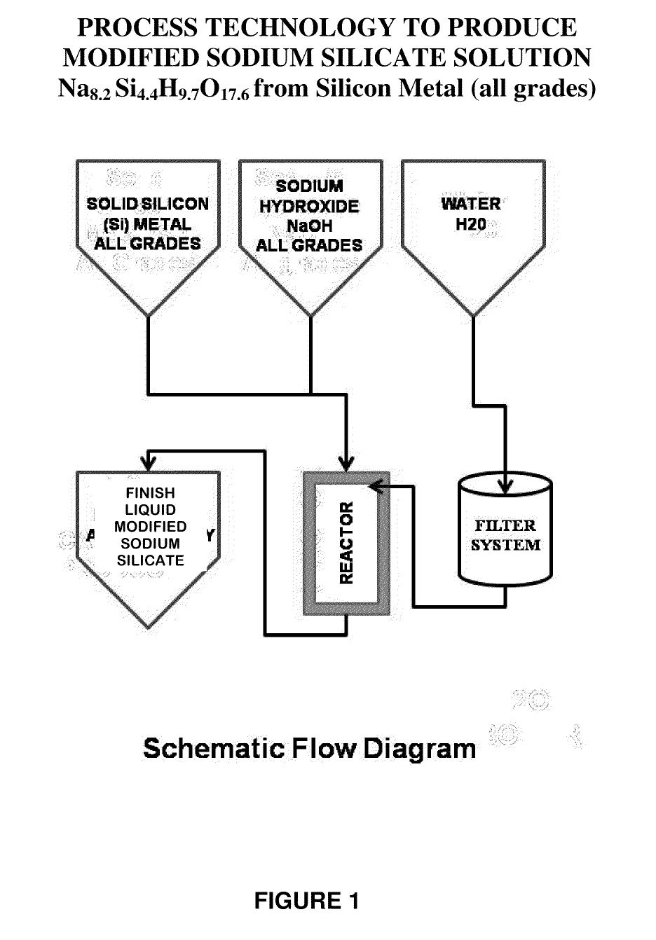

To produce modified sodium silicate, silicon metal (any grade) is loaded into a reactor. Sodium hydroxide is added along with water. An exothermic reaction occurs. The reaction is allowed to proceed for 4-6 hours, after which the product is collected in a cooling tank. The product is cooled and the obtained liquid product is packaged. FIG. 1 is a schematic flow diagram of the process for making the composition.

In one embodiment, the ingredients for preparing modified sodium silicate are as follows:

about 1-10 parts silicon metal (all grades);

about 1-10 parts sodium hydroxide (all grades); and

about 5-20 parts water.

Silicon metal (any grade) from source 1 is loaded into reactor 5. Then 1-10 parts of sodium hydroxide from source 2 is loaded into the reactor 5, and 5-20 parts of water from source 3 are added through a filtration system. An exothermic reaction occurs, which is allowed to continue for about four to six hours. The product is removed from the silicon and collected as a liquid in a cooling tank 4 and cooled at ambient room temperature. Water can then be added to reach a specific density of the liquid product. The liquid product can be further filtered. The product obtained was an aqueous solution with an empirical formula of Na_{8.2}Si_{4.4}H_{9.7}O_{17.6}. At this point, the resultant product is in aqueous form and it is ready for packaging and use. It is non-toxic and not corrosive.

In a preferred embodiment for making the product of the instant application, the silicon used according to the present process is preferably silicon rock of 97-99% purity. Impurities can be less than 1% iron and less than 1% aluminum. The sodium hydroxide solution can have a specific gravity of from 1.11 to 1.53 and can contain from about 40 to about 50% by weight sodium hydroxide.

In a preferred embodiment, the silicon added to the reactor and used in the process to make modified sodium silicate is in rock form (specific gravity of 2.3) and preferably the amount used is in the range of about 40 to 350 pounds, and more preferably in a range of about 46.7 to 300 pounds.

The amount of water used is in the range of about 5.0 to 35 gallons, and more preferably in a range of about 5.5 to 29.8 gallons, and this water is pre-heated to a temperature of about 140-150° F.

The amount of the sodium hydroxide (grade 50) used is in a range of about 1.0 to 15.00 gallons, and more preferably in a range of about 2.05 to 11.18 gallons.

As discussed above, the silicon-based alkaline composition (modified sodium silicate) of the present application is not a single compound but an aqueous mixture of the following two compounds in equilibrium with each other:

1. Trimeric Sodium Silicate (Na₂SiO₃)₃, shown in FIG. 19; and

2. Sodium Silicate Pentahydrate (Na₂SiO₃).5H₂O, shown in FIG. 20.

Preferably, the silicon-based alkaline composition (empirical formula of Na_{8.2}Si_{4.4}H_{9.7}O_{17.6}) has a specific density in the range of 1.24 to 1.26, and more preferably the specific density is 1.25+/−. The composition also has a pH in the range of 13.8 to 14.0, and preferably it is 13.9+/−.

Many of the biological activities of modified sodium silicate could be due to the multiple ionized forms, giving it the ability to accept and donate electrons and participate in important redox reactions in the body to bring about redox homeostasis. The elemental and chemical properties of modified sodium silicate give it unique electrochemical and structural characteristics to participate in reactions in cancer cells and the HIV-virus that are beneficial. These health promoting properties of modified sodium silicate appear to be directly related to ways it regulates redox processes of biological molecules through different free radical species of oxygen and nitrogen.

Changes in redox status appear to lead to unknown ways in which cellular and viral biochemical systems are then modulated. Work with cancer models (as discussed herein below) has given empirical evidence that this compound has an anticancer effect. It has been noted in these studies that modified sodium silicate can reduce tumor size, reduce remissions and aid in chemotherapy by increasing effectiveness and reducing side effects.

Preliminary studies have also shown an anti-retroviral effect for modified sodium silicate as discussed further below.

Accordingly, one embodiment of the present application provides for a method for treating cancer in a subject in need thereof by causing one or more anti-cancer effects by administering to the patient an effective amount of the silicon-based alkaline composition as described herein, wherein the one or more anti-cancer effects are selected from the group consisting of preventing attachment of cancer cells; reducing harmful mutations in cellular DNA; inducing apoptosis; and stimulating anti-oxidant enzymes. In another embodiment, the present application provides for a method of preventing attachment of cancer cells; a method of reducing harmful mutations in cellular DNA; a method inducing apoptosis; and a method of stimulating anti-oxidant enzymes.

The cancers that can be treated or prevented by the composition and method of the present application include, but are not limited to, human sarcomas and carcinomas, and lymphomas, e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon cancer, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, retinoblastoma; leukemias, e.g., acute lymphocytic leukemia and acute myelocytic leukemia (myeloblastic, promyelocytic, myelomonocytic, monocytic and erythroleukemia); chronic leukemia (chronic myelocytic (granulocytic) leukemia and chronic lymphocytic leukemia); and polycythemia vera, lymphoma (Hodgkin's disease and non-Hodgkin's disease), multiple myeloma, Waldenstrobm's macroglobulinemia, and heavy chain disease.

In a preferred embodiment, the method of the present application provides for treating colon cancer.

In another embodiment, the present application provides for a method for treating viral infection in a subject in need thereof by causing one or more anti-viral effects by administering to the patient an effective amount of the silicon-based alkaline composition as described herein, wherein the one or more anti-viral effects are selected from the group consisting of increasing nitric oxide dependent anti-viral effects; inhibiting enzymes involved in viral assembly; causing changes in viral carbohydrate composition; and inhibiting viral enzymes responsible for transcribing RNA to DNA.

Preferably, the viral infections to be treated or prevented by the composition and method of the present application include, but are not limited to, retroviral infection. Thus, the present application provides for treatment of retroviruses, which are generally defined as any of a group of viruses (many of which produce tumors, including the virus that causes AIDS) whose genetic information is contained in RNA, as opposed to DNA. Retroviruses contain reverse enzyme reverse transcriptase for generating DNA from RNA.

Examples of retroviruses to be treated by the present application include those belonging to the following: Genus Alpharetrovirus, type species: Avian leucosis virus; Genus Betaretrovirus; type species: Mouse mammary tumour virus; Genus Gammaretrovirus, type species: Murine leukemia virus, others include Feline leukemia virus; Genus Deltaretrovirus; type species: Bovine leukemia virus, others include Human T-lymphotropic virus; Genus Epsilonretrovirus; type species: Walleye dermal sarcoma virus; Genus Lentivirus; type species: Human immunodeficiency virus 1, others include Simian and Feline immunodeficiency viruses; Genus Spumavirus, type species: Chimpanzee foamy virus. In some embodiments, the virus is a retrovirus derived from a avian sarcoma and leukosis retroviral group, a mammalian B-type retroviral group, a human T cell leukemia and bovine leukemia retroviral group, a D-type retroviral group, a murine leukemia-related group, or a lentivirus group. Often, the virus a lentivirus. In particular embodiments, the retrovirus is an HIV-1, an HIV-2, an SIV, a BIV, an EIAV, a Visna, a CaEV, an HTLV-1, a BLV, an MPMV, an MMTV, an RSV, a FeLV, a BaEV, or an SSV retrovirus. Preferably, the retrovirus is HIV-1 or HIV-2.

Preferably, the viral infection is a retroviral infection, for example, an infection caused by the human immunodeficiency virus (HIV).

In the body, the virus evades the immune system and attaches to cells using surface sugars. Different sugars on the virus determine the shape of the virus. If these sugars can be changed, the shape of the virus is changed so that it can no longer evade the immune system, and can no longer bind to cell receptors.

Glucohydrolase enzymes are found in the Golgi apparatus of the host's cells. Inhibition of these enzymes has been found to decrease the infectivity of viral enzyme virions. Glucosidase and glucoronidase add sugars to the viral envelope, thus changing the configuration of the virus and inhibiting its binding to cell surface receptors.

The modified sodium silicate has been found to increase nitric oxide dependent ant-viral effects at all concentrations tested. Modified sodium silicate has also been found to inhibit enzymes that are important in viral assembly, metabolism and replication. Modified sodium silicate has caused changes in the viral carbohydrate composition and metabolism, and inhibited the activity of the enzyme responsible for transcribing RNA and DNA in the virus; these effects were dose dependent. In addition, modified sodium silicate inhibited reverse transcriptase activity, completely inhibiting establishment or reproduction of retroviruses in vivo.

Viral proteases are involved in the process of facilitating the production of new viruses. In the absence of viral protease activity, viral assembly is unlikely. The modified sodium silicate inhibited viral protease activity by 62% in laboratory research.

Modified sodium silicate has been found to increase nitric oxide dependent anti-viral effects at all concentrations tested, and has been found to inhibit enzymes important in viral assembly, metabolism, and replication.

Compositions within the scope of the present invention include all compositions wherein the modified sodium silicate is contained in an amount effective to achieve its intended purpose. The term “effective amount” as used herein means an amount effective, at dosages and for periods of time necessary to achieve the desired result. While individual needs vary, determination of optimal ranges of effective amounts of each compound is within the skill of the art. Typical dosages comprise 0.01 to 100 mg/kg body weight. The preferred dosages comprise 0.1 to 100 mg/kg body weight. The most preferred dosages comprise 1 to 50 mg/kg body weight.

Pharmaceutical compositions for administering modified sodium silicate preferably contain, in addition to the modified sodium silicate, suitable pharmaceutically acceptable carriers comprising excipients and auxiliaries which facilitate processing of the modified sodium silicate into preparations which can be used pharmaceutically. Preferably, the preparations, contain from about 0.01 to about 99 percent by weight, preferably from about 20 to 75 percent by weight, modified sodium silicate, together with the excipient. For purposes of the present invention, all percentages are by weight unless otherwise indicated.

The pharmaceutically acceptable carriers include vehicles, adjutants, excipients, or diluents that are well known to those skilled in the art and which are readily available. It is preferred that the pharmaceutically acceptable carrier be one which is chemically inert to modified sodium silicate and which has no detrimental side effects or toxicity under the conditions of use.

There is a wide variety of suitable formulations of the pharmaceutical compositions of the present invention. Formulations can be prepared for oral, aerosol, parenteral, subcutaneous, intravenous, submucosal transdermal, intra arterial, intramuscular, intra peritoneal, intra tracheal, rectal, and vaginal administration.

Other pharmaceutically acceptable carriers for the active ingredients according to the present invention are liposomes, pharmaceutical compositions in which the modified sodium silicate is contained either dispersed or variously present in corpuscles consisting of aqueous concentric layers adherent to lipid layers. The modified sodium silicate may be present both in the aqueous layer and in the lipid layer, inside or outside, or, in any event, in the nonhomogeneous system generally known as a liposomic suspension. The hydrophobic layer, or lipid layer, generally, but not exclusively, comprises phospholipids such as lecithin and sphingomyelin, steroids such as cholesterol, more or less ionic surface active substances such as dicetyl phosphate, stearylamine, or phosphatidic acid, and/or other materials of a hydrophobic nature.

Modified sodium silicate may also be formulated for transdermal administration, for example in the form of transdermal patches so as to achieve systemic administration.

Formulations suitable for oral administration, including submucosal and transbuccal, can consist of liquid solutions such as effective amounts of modified sodium silicate dissolved in diluents such as water, saline, or orange juice; capsules, tables, sachets, lozenges, and troches, each containing a predetermined amount of the active ingredient as solids or granules; powders, suspensions in an appropriate liquid; and suitable emulsions. Liquid formulations may include diluents such as water and alcohols, e.g., ethanol, benzyl alcohol, and the polyethylene alcohols, either with or without the addition of a pharmaceutically acceptable surfactant, suspending agents, or emulsifying agents. Capsule forms can be of the ordinary hard- or soft-shelled gelatin type containing, for example, surfactants, lubricant, and inert fillers, such as lactose, sucrose, calcium phosphate, and corn starch. Tablet forms can include on e or more of lactose, sucrose, mannitol, corn starch, potato starch, alginic acid, microcrystalline cellulose, acacia, gelatin, guar gum, colloidal silicon dioxide, croscaramellose sodium, talc, magnesium stearate, calcium stearate, zinc stearate, stearic acid, and other preservatives, flavoring agents, and pharmaceutically acceptable disintegrating agents, moistening agents preservatives flavoring agents, and pharmacologically compatible carriers. Lozenge forms can comprise modified sodium silicate in a carrier, usually sucrose and acacia or tragacanth, as well as pastilles comprising modified sodium silicate in an inert base such as gelatin or glycerin, or sucrose and acacia. Emulsions and the like can contain, in addition to modified sodium silicate, such carriers as are known in the art.

Formulations suitable for parenteral administration include aqueous and non-aqueous, isotonic sterile injection solutions, which can contain anti-oxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. The modified sodium silicate can be administered in a physiologically acceptable diluent in a pharmaceutical carriers, such as a sterile liquid or mixture of liquids, including water, saline, aqueous dextrose and related sugar solutions, an alcohol such as ethanol, isopropanol, or hexadecyl alcohol, glycols such as propylene glycol or polyethylene glycol, glycerol ketals such as 2,2-dimethyl-1,3-dioxolane-4-methanol, ethers such as poly(ethylene glycol) 400, oils, fatty acids, fatty acid esters or glycerides, or acetylated fatty acid glycerides, without the addition of a pharmaceutically acceptable surfactants, such as soap or a detergent, suspending agent, such as carbomers, methylcellulose, hydroxypropylmethylcellulose, or carboxymethylcellulose, or emulsifying agents and other pharmaceutical adjutants.

Oils which can be used in parenteral formulations include petroleum, animal, vegetable, or synthetic oils. Specific examples of oils include peanut, soybean, sesame, cottonseed, corn, olive, petrolatum, and mineral. Fatty acids can be used in parenteral formulations, including oleic acid, stearic acid, and isostearic acid. Ethyl oleate and isopropyl myristate are examples of suitable fatty acid esters. Suitable salts for use in parenteral formulations include fatty alkali metal, ammonium, and triethanolamine salts, and suitable detergents include cationic detergents such as dimethyl dialkyl ammonium halides, and alkyl pyridimium halides; anionic detergents such as dimethyl olefin sulfonates, alkyl, olefin, ether, and monoglyceride sulfates and sulfosuccinates, polyoxyethylenepolypropylene copolymers; amphoteric detergents such as alkyl-beta-aminopropionates and 2-alkyl-imidazoline quaternarry ammonium salts; and mixtures thereof.

Additionally, modified sodium silicate can be formulated into suppositories by mixing the active ingredient with a variety of bases, including emulsifying bases or water-soluble bases. Formulations suitable for vaginal administration may be in the form of pessaries, tampons, creams, gels, pastes, foam, or spray formulations containing, in addition to the active ingredient, such carriers as are known in the art to be appropriate.

Any number of assays well known in the art may be used to demonstrate that modified sodium silicate can be used for treating cancer cells, treating retroviral infections, increasing the amount of nitric oxide in the body, or treating oxidative stress by reducing reactive oxygen species in the body, or treating oxidative stress by reducing reactive oxygen species in the body.

In determining the dosages of modified sodium silicate to be administered, the dosage and frequency of administration is selected in relation to the pharmacological properties of the specific active ingredients. Normally, at least three dosage levels should be used. In toxicity studies in general, the highest dose should reach a toxic level but be sub lethal for most animals in the group. If possible, the lowest dose should induce a biologically demonstrable effect. These studies should be performed in parallel for each compound selected.

Additionally, the ID₅₀ level of modified sodium silicate in question can be one of the dosage levels selected, and the other two selected to reach a toxic level. The lowest dose that dose not exhibit a biologically demonstrable effect. The toxicology tests should be repeated using appropriate new doses calculated on the basis of the results obtained. Young, healthy mice or rats belonging to a well-defined strain are the first choice of species, and the first studies generally use the preferred route of administration. Control groups given a placebo or which are untreated are included in the tests. Tests for general toxicity, as outlined above, should normally be repeated in another non-rodent species, e.g., a rabbit or dog. Studies may also be repeated using alternate routes of administration.

Single dose toxicity tests should be conducted in such a way that signs of acute toxicity are revealed and the mode of death determined. The dosage to be administered is calculated on the basis of the results obtained in the above-mentioned toxicity tests. Data on single dose toxicity, e.g., ID₅₀, the dosage at which half of the experimental animals die, is to be expressed in units of weight or volume per kg of body weight and should generally be furnished for at least two species with different modes of administration. In addition to the ID₅₀ value in rodents, it is desirable to determine the highest tolerated dose and/or lowest lethal dose for other species, i.e., dog and rabbit.

When a suitable and presumably safe dosage level has been established as outlined above, studies on the drug's chronic toxicity, its effect on reproduction, and potential mutagenicity may also be required in order to ensure that the calculated appropriate dosage range will be safe, also with regard to these hazards.

Pharmacological animal studies on pharmacokinetics revealing, e.g., absorption, distribution, biotransformation, and excretion of the active ingredient and metabolites are then performed. Using the results obtained, studies on human pharmacology are then designed. Studies of the pharmacodynamics and pharmacokinetics of the compounds in humans should be performed in healthy subjects using the routes of administration intended for clinical use, and can be repeated in patients. The dose-response relationship when different doses are given, or when several types of conjugates or combinations of conjugates and free compounds are given, should be studied in order to elucidate the dose-response relationship (dose vs. plasma concentration vs. effect), the therapeutic range, and the optimum dose interval. Also, studies on time-effect relationship, e.g., studies into the time-course of the effect and studies on different organs in order to elucidate the desired and undesired pharmacological effects of the drug, in particular on other vital organ systems, should be performed.

The amount of modified sodium silicate to be administered to any given patient must be determined empirically, and will differ depending upon the condition of the patients. Relatively small amounts of the active ingredient can be administered at first, with steadily increasing dosages if no adverse effects are noted. Of course, the maximum safe toxicity dosage as determined in routine animal toxicity tests should never be exceeded.

The preferred patients/animal subjects to be treated are mammals, and preferably humans.

“Treating” a disease state or condition refers to an approach for obtaining beneficial or desired effects and results, including clinical results. Beneficial or desired clinical results can include, but are not limited to, alleviation or amelioration of one or more symptoms or conditions, diminishment of extent of disease, stabilization of the state of disease, prevention of development of disease, prevention of spread of disease, delay or slowing of disease progression, delay or slowing of disease onset, amelioration or palliation of the disease state, and remission (whether partial or total), whether detectable or undetectable. “Treating” can also mean prolonging survival of a patient beyond that expected in the absence of treatment. “Treating” can also mean inhibiting the progression of disease, slowing the progression of disease temporarily, although more preferably, it involves halting the progression of the disease permanently. As will be understood by a skilled person, results may not be beneficial or desirable if, while improving a specific disease state, the treatment results in adverse effects on the patient treated that outweigh any benefits effected by the treatment.

For the treatment of cancer, the modified sodium silicate may be administered one or more times, and optionally, it may be used in combination with one or more other anti-cancer therapies and/or anti-cancer agents, including, for example, radiation therapy, chemotherapy, surgery, hyperthermia, laser, photodynamic therapy, inhibition of angiogenesis, bone marrow transplantation, and gene therapy. Some representative examples of anti-cancer agents are discussed below.

Examples of anti-cancer chemotherapeutic agents include: alkylating antineoplastic agents. Alkylating agents are so named because of their ability to add alkyl groups to many electronegative groups under conditions present in cells. Cisplatin and carboplatin, as well as oxaliplatin, are alkylating agents. They impair cell function by forming covalent bonds with the amino, carboxyl, sulfhydryl, and phosphate groups in biologically important molecules. Other agents are mechlorethamine, cyclophosphamide, chlorambucil, ifosfamide. They work by chemically modifying a cell's DNA.

Other anti-cancer agents include anti-metabolites masquerading as purine ((azathioprine, mercaptopurine)) or pyrimidine—which become the building blocks of DNA. They prevent these substances from becoming incorporated in to DNA during the “S” phase (of the cell cycle), stopping normal development and division. They also affect RNA synthesis. Due to their efficiency, these drugs are the most widely used cytostatics.

Other anti-cancer agents include alkaloids. Alkaloids, which can be derived from plants, block cell division by preventing microtubule function. Microtubules are vital for cell division, and, without them, cell division cannot occur. The main examples are vinca alkaloids and taxanes. Vinca alkaloids bind to specific sites on tubulin, inhibiting the assembly of tubulin into microtubules (M phase of the cell cycle). They are derived from the Madagascar periwinkle, Catharanthus roseus (formerly known as Vinca rosea). The vinca alkaloids include: Vincristine, Vinblastine, Vinorelbine, and Vindesine.

Another anti-cancer agent is podophyllotoxin, a plant-derived compound which is said to help with digestion as well as used to produce two other cytostatic drugs, etoposide and teniposide. They prevent the cell from entering the G1 phase (the start of DNA replication) and the replication of DNA (the S phase). The substance has been primarily obtained from the American Mayapple (Podophyllum peltatum). Recently it has been discovered that a rare Himalayan Mayapple (Podophyllum hexandrum) contains it in a much greater quantity, but, as the plant is endangered, its supply is limited.

Other anti-cancer agents include taxanes. The prototype taxane is the natural product paclitaxel, marked as TAXOL® by Bristol-Myers Squibb Corporation and first derived from the bark of the Pacific Yew tree. Docetaxel is a semi-synthetic analogue of paclitaxel. Taxanes enhance stability of microtubules, preventing the separation of chromosomes during anaphase.

Other anti-cancer agents include topoisomerases, which are essential enzymes that maintain the topology of DNA. Inhibition of type I or type II topoisomerases interferes with both transcription and replication of DNA by upsetting proper DNA supercoiling. Some type I topoisomerase inhibitors include camptothecins: irinotecan and topotecan. Examples of type II inhibitors include amsacrine, etoposide, etoposide phosphate, and teniposide. These are semisynthetic derivatives of epipodophyllotoxins, alkaloids naturally occurring in the root of American Mayapple (Podophyllum peltatum).

Antitumour antibiotics may also be used, which include, for example, actinomycin, dactinomycin, anthracyclines (e.g., doxorubicin, daunorubicin. Valrubicine, Idarubicine, epirubicin). Other cytotoxic antibiotics include, for example, bleomycin, plicamycin, mitomycin.

The dosages of prophylactic or therapeutic agents other than modified sodium silicate, which have been or are currently being used to prevent, treat, manage, or proliferative disorders, such as cancer, or one or more symptoms thereof can be used in the combination therapies of the invention. The recommended dosages of agents currently used for the prevention, treatment, management, or amelioration of a proliferative disorders, such as cancer, or one or more symptoms thereof, can obtained from any reference in the art including, but not limited to, Hardman et al., eds., 1996, Goodman & Gilman's The Pharmacological Basis Of Basis Of Therapeutics 9.sup.th Ed, Mc-Graw-Hill, New York; Physician's Desk Reference (PDR) 57.sup.th Ed., 2003, Medical Economics Co., Inc., Montvale, N.J., which are incorporated herein by reference in its entirety.

In certain embodiments, when the modified sodium silicate is administered in combination with another therapy, the therapies (e.g., prophylactic or therapeutic agents) are administered less than 5 minutes apart, less than 30 minutes apart, 1 hour apart, at about 1 hour apart, at about 1 to about 2 hours apart, at about 2 hours to about 3 hours apart, at about 3 hours to about 4 hours apart, at about 4 hours to about 5 hours apart, at about 5 hours to about 6 hours apart, at about 6 hours to about 7 hours apart, at about 7 hours to about 8 hours apart, at about 8 hours to about 9 hours apart, at about 9 hours to about 10 hours apart, at about 10 hours to about 11 hours apart, at about 11 hours to about 12 hours apart, at about 12 hours to 18 hours apart, 18 hours to 24 hours apart, 24 hours to 36 hours apart, 36 hours to 48 hours apart, 48 hours to 52 hours apart, 52 hours to 60 hours apart, 60 hours to 72 hours apart, 72 hours to 84 hours apart, 84 hours to 96 hours apart, or 96 hours to 120 hours part. In one embodiment, two or more therapies (e.g., prophylactic or therapeutic agents) are administered within the same patient visit.

In certain embodiments, the modified sodium silicate and one or more other the therapies (e.g., prophylactic or therapeutic agents) are cyclically administered. Cycling therapy, involves the administration of a first therapy (e.g., a first prophylactic or therapeutic agents) for a period of time, followed by the administration of a second therapy (e.g., a second prophylactic or therapeutic agents) for a period of time, followed by the administration of a third therapy (e.g., a third prophylactic or therapeutic agents) for a period of time and so forth, and repeating this sequential administration, i.e., the cycle in order to reduce the development of resistance to one of the agents, to avoid or reduce the side effects of one of the agents, and/or to improve the efficacy of the treatment.

In certain embodiments, administration of the same compound of the invention may be repeated and the administrations may be separated by at least 1 day, 2 days, 3 days, 5 days, 10 days, 15 days, 30 days, 45 days, 2 months, 75 days, 3 months, or 6 months. In other embodiments, administration of the same prophylactic or therapeutic agent may be repeated and the administration may be separated by at least at least 1 day, 2 days, 3 days, 5 days, 10 days, 15 days, 30 days, 45 days, 2 months, 75 days, 3 months, or 6 months.

For use as an anti-viral agent, the silicon-based alkaline composition may be administered one or more times, and, optionally, it may be used in combination with one or more vaccines, or other anti-viral agents and/or anti-viral therapies.

Examples of other anti-viral agents include:

Abacavir, Aciclovir, Acyclovir, Adefovir, Amantadine, Amprenavir, Arbidol, Atazanavir, Atripla, Boceprevir, Cidofovir, Combivir, Darunavir, Delavirdine, Didanosine, Docosanol, Edoxudine, Efavirenz, Emtricitabine, Enfuvirtide, Entecavir, Entry inhibitors, Famciclovir, Fomivirsen, Fosamprenavir, Foscarnet, Fosfonet, Fusion inhibitor, Ganciclovir, Ibacitabine, Immunovir, Idoxuridine, Imiquimod, Indinavir, Inosine, Integrase inhibitor, Inteferon types I-III, Lamivudine, Lopinavir, Loviride, Maraviroc, Moroxydine, Nelfinavir, Nevirapine, Nexavir, Nucleoside analogues, Oseltamivir (Tamiflu), Peginterferon alfa-2a, Penciclovir, Peramivir, Pleconaril, Podophyllotoxin, Protease inhibitor (pharmacology), Raltegravir, Reverse transcriptase inhibitor, Ribavirin, Rimantadine, Ritonavir, Saquinavir, Stavudine, Synergistic enhancer (antiretroviral), Tenofovir, Tenofovir disoproxil, Tipranavir, Trifluridine, Trizivir, Tromantadine, Truvada, Valaciclovir, Valganciclovir, Vicriviroc, Vidarabine, Viramidine, Zalcitabine, Zanamivir, Zidovudine

In a preferred embodiment, examples of one or more other anti-retroviral drugs are compounds selected from lamivudine, zidovudine, stavudine, abacavir, adefovir, tenofovir, emtricitabine, zalcitabine, didanosine, efavirenz, nevirapine, delavirdine, indinavir, nelfinavir, lopinavir, ritonavir, saquinavir, amprenavir, atazanavir, tipranavir, fosamprenavir or mixtures thereof. Preferred anti-retroviral drugs in such water-dispersible compositions include lamivudine, stavudine, nevirapine or mixtures thereof.

The term “anti-retroviral drugs,” as used herein includes drugs or compounds intended for treating, reversing, reducing or inhibiting retroviral infections, in particular infections caused by HIV. The anti-retroviral drug may be selected from various classes of drugs, such as nucleoside or non-nucleoside reverse transcriptase inhibitors or protease inhibitors. Nucleoside reverse transcriptase inhibitors may include lamivudine, zidovudine, stavudine, abacavir, adefovir, tenofovir, emtricitabine, zalcitabine and didanosine. Non-nucleoside reverse transcriptase inhibitors may include efavirenz, nevirapine and delavirdine. Protease inhibitors may include indinavir, nelfinavir, lopinavir, ritonavir, saquinavir, amprenavir, atazanavir, tipranavir and fosamprenavir. Anti-retroviral drugs includes free base, as well as pharmaceutically acceptable salts, solvates, enantiomers, esters or polymorphs thereof or any compound, which upon administration to the recipient, is capable of providing the anti-retroviral drug or any active metabolite or residue thereof, either directly or indirectly.

### Anti-Cancer Activity:

As discussed and shown herein, work with cancer models has given empirical evidence that that the silicon-based alkaline composition of the present application has an anti-cancer effect. It has been noted in these studies that modified sodium silicate can reduce tumor size, reduce remissions and aid in chemotherapy by increasing effectiveness and reducing side effects. By way of this application, the inventors have shown that modified sodium silicate as the following anti-cancer effects:
modified sodium silicate prevented attachment of cancer cells in a dose-dependent manner;
modified sodium silicate reduced harmful mutations in the DNA and its effects were dose-dependent;
modified sodium silicate induced apoptosis (programmed cell death) of and its effects were time and dose-dependent; and
modified sodium silicate stimulated important antioxidant enzymes in a dose dependent manner.

### Materials and Methods:

### Determination of Antimutagenic Activity

Salmonella typhimurium (TA 100, 98, 1535, 1537 and 1538) cultures were grown overnight in Nutrient Broth. Voges-Bonner medium with 1.5% agar was used as the bottom agar. The top agar overlay consisted of 0.6% agar with trace amounts of biotin and histidine. Antimutagen was poured into Petri plates, containing 20 ml of agar a 3 ml of top soft agar overlay mixed with 0.1 ml each of bacteria, mutagen. The plates were then incubated at 37° C. for 48 hours. The number of colony forming units (c.f.u.) after incubation were counted. Controls for spontaneous reversions, mutagen and antimutagen treatments were run along with the evaluated treatments in various combinations mentioned previously. Mutagen: Different concentrations of NaN₃ (in distilled water) were added to the top agar (Ames et al 2003) to give 1, 2 and 5 μg per plate. Antimutagen: modified sodium silicate in water, diluted and then added to top agar medium (Ames et al 2003).

### Cells and Culture

Colon cancer cell line (HT-29) was purchased from ATCC, Manassas, Va., USA and used between passages 3-25 for all experiments. Cells were grown as monolayers in Dulbecco's modified eagle medium (DMEM), 4500 mg/L glucose (Gibco, Life Technologies Ltd, UK) with 10% fetal calf serum (FCS), 2 mmol/L Lglutamine, 60 μ/mL penicillin and 60 μg/mL streptomycin, in a humidified atmosphere of 95% air and 5%/CO₂ at 37° C.

### Cytotoxicity Assay

An ALMAR BLUE proliferation kit (Almar, Sacramento, Calif., USA) was used to measure anti-proliferative effects in all the cells. For the cytotoxicity assay, the cells (30,000 cells/well) were seeded onto a 96-multiwell plate together with DMEM (10% FCS) and incubated overnight at 37° C. at 5%/CO₂. The day after, the cells were washed with PBS and treated with modified sodium silicate (2-4 μM) diluted in DMEM (0.1% FCS) by the adding 100 μl of the extracts to the wells for each condition. Every experiment was performed in octuple and 500 μM deoxycholic acid was used as a positive control. After 4 days of incubation (37° C. at 5% CO₂) Almar blue dye (10%) was added and the cells were placed in the same incubator. After 5-6 hours, the plates were read at 570 nm in a microplate spectrophotometer (Biot-Tek 808 IUC) (Bio-Tek Instrument, VT). Cell survival was expressed as the percentage absorbance of the mean absorbance of the negative control (DMEM 0.1% FCS).

### Adhesion Assay

Colon cancer cell line (HT-29) was purchased from ATCC, Manassas, Va., USA and used between passages 3-25 for all experiments. Cells were grown as monolayers in Dulbecco's modified eagle medium (DMEM), 4500 mg/L glucose (Gibco, Life Technologies Ltd, UK) with 10% fetal calf serum (FCS), 2 mmol/L Lglutamine, 60 U/mL penicillin and 60 μg/mL streptomycin, in a humidified atmosphere of 95% air and 5% CO₂ at 37° C. Cells were split and seeded at 5×10⁶ with different concentrations of modified sodium silicate in a 8 well plate and incubated in a humidified atmosphere of 95% air and 5% CO₂ at 37° C. for 24 hours. After 24 hours the plates were washed to remove non-adherent cells and the adherent cells were trypsinized and counted under a hemocytometer.

### Assays for Effects on Apoptosis

Evasion of normal apoptotic process is involved in tumorigenesis and therefore the effect of modified sodium silicate on induction of apoptosis was investigated.

### DNA Fragmentation Analysis

This assay used centrifugal sedimentation to separate fragmented double-stranded DNA from intact DNA. Upon lysis of cells, cytosolic DNA is released and a centrifugation step will generate two fractions corresponding to intact and fragmented DNA (present in cytosol). Acid hydrolysis allows for deoxyribose sugars to bind with DNA, and the percentage of fragmented DNA can be quantified spectrophotometrically. Amount of fragmented DNA is directly proportional to apoptotic activity. The cell pellets (5×10⁶) were lysed in 0.5 ml of lysis buffer containing 5 mM Tris-HCl, 20 mM ethilenediaminetetraacetic acid (EDTA) and 0.5% Triton X 100. After centrifugation at 1,500×g for 10 minutes, the pellets were resuspended in 250 μL of lysis buffer and, to the supernatants (S), 20 μL of 6 M perchloric acid was added. Then, 500 μL of 10% trichloroacetic acid (TCA) were added to the pellets (P). The samples were then centrifuged for 10 min at 5,000 rpm and the pellets were resuspended in 250 μL of 5% TCA followed by incubation at 100° C. for 15 minutes. Subsequently, to each sample, 500 μL of solution (15 mg/ml DPA in glacial acetic acid), 15 μL/ml of sulfuric acid and 15 μg/ml acetaldehyde were added and incubated at 37° C. for 18 hours (20). The proportion of fragmented DNA was calculated from the absorbance at 594 nm using the following formula: Fragmented DNA (%)=100×(amount of the fragmented DNA in the supernatant)/(amount of the fragmented DNA in the supernatant+amount DNA in the pellets).

### Malondialdehyde (MDA) Assay

Malondialdehyde was measured by modifying the method discussed by Tamagnone et al., 1998 (35). In a test tube 200 μl of the briefly treated and untreated cell homogenate was mixed with 800 μl of water, 500 μl of 20% (w/v) trichloroacetic acid and 1 ml of 10 mM thiobarbutyric acid. The test tubes were incubated for 30 minutes at 100° C. and then centrifuged at 13,000 rpm for 10 minutes. The absorbance of the supernatant was measured at 532 nm and the concentration of MDA was calculated from its molar extinction coefficient (ε) 156 μmol⁻¹cm⁻¹ and expressed as μmmol/g FW.

### SOD-Riboflavin-NBT Assay

The SOD activity was measured by its ability to prevent superoxide mediated oxidation of NBT to Diformazan as a result of the photooxidation of riboflavin. Briefly, 20 μL of treated and untreated cell suspension was transferred into each well of a 96 well plate. 150 μL of riboflavin reaction mixture (2 mM riboflavin, 50 mM KH₂PO₄ buffer (pH 8.0), 0.1 mM EDTA, 200 μM DTPA and 57 μM NBT) was transferred to the well. Then, 170 μL of riboflavin reaction mixture was added to a well to serve as the blank. Plates were incubated in dark in a chamber which exposed the 96 well plate to fluorescent lamps for 20 minutes and absorbance was read at 560 nm. Calculation of the concentration of Diformazan was determined using its molar extinction coefficient, 26478 mol-1 cm⁻¹. The concentration of Diformazan was expressed as μmol/mg of protein.

### Glutathione Determination Assay

The concentration of reduced glutathione in cells was determined after treatment of the cells for 24 hours. The cells (3×10⁵/ml) were washed with physiological solution and lysed with water; 3 ml of precipitant solution (1.67 g glacial metaphosphoric acid, 0.2 g ethylenediaminetetraacetic acid (EDTA) and 30 g NaCl in 100 ml MilliQ water) were added to the lysate (2 ml). After 5 minutes, this mixture was centrifuged and 0.4 ml of the supernatant was added to 1.6 ml of reaction medium (0.2M Na₂HPO₄ buffer, pH 8.0; 0.5 mM DTNB dissolved in 1% sodium citrate). Subsequently, the absorbance of the product (NTB) was measured at 412 nm and reduced glutathione concentration calculated using the extinction coefficient E=13.6 mol-1 cm⁻¹ (19).

### Antioxidant Systems

Secondary to the necessary production of reactive oxygen species ROS in vivo, the body must provide a mechanism for removal of excess ROS to prevent the damaging effects of oxidation. Mechanisms for ROS removal include primary, secondary, and tertiary antioxidant defenses. Primary antioxidant defense prevents oxidation by ROS; primary defense encompasses specific antioxidant enzymes, including superoxide dismutase (SOD), catalase (CAT), glutathione peroxidase (GP), and glutathione transferase (GT). Proteins, DNA, and lipids are oxidized upon formation of excess ROS which leads to decreased protein activity, improper protein activity, transcription factor activation, and abnormal and untimely cell proliferation and apoptosis. Proteins that are especially susceptible to oxidation include those that contain the amino acids cysteine, methionine, arginine, histidine, tryptophan, and tyrosine. DNA oxidation occurs upon hydrogen abstraction, which causes strands of DNA to break, crosslink, or alterations in base modification. Base modification alterations occur upon hydrogen abstraction; this causes the DNA repair system, which proofreads copies of DNA, to misread the copied DNA. The DNA copy is misread by the DNA repair system because, when the DNA copy is oxidized, it is unrecognizable to the repair system. This leads to incorrect pairing of DNA bases, which leads to DNA mutation and possibly cancer.

### Anti-Mutagenic Effects

The test compound, modified sodium silicate, was serially diluted in distilled water and filter sterilized. The silicates were quantified by the ammonium molybdate assay at 450 nm. MAS-NMR and FT-IR were used for structural determination. The anti-mutagenic effects were determined using Ames test trans and NaN₃ as a mutagen. HT-29 cells were cultured using standard protocols and were seeded at 10⁶ cells/well with different concentrations of sterile active compound. L. terrestris was cultured using standard culture techniques.

Cell survival was assayed using Trypan blue exclusion enumeration.

Anti-adhesive effects were evaluated in treated 96-well plates counting attached cells after 24 hours. MDA concentration was determined by its reaction with thiobarbituric acid (TBA) at 532 nm. SOD activity was determined by assaying by the NBT. Diformazan assay qt 560 nm. CAT activity was determined by measuring the formation of chromic acetate from dichromate at 570 nm. Protein was measured using the Bradford assay. GSH content was determined by assaying for the GSH-DTNB (Ellman's reagent) qt 417 nm.

The modified sodium silicate was used at a concentration of 0.37M. The antimutagenic effects were observed with test compounds in various Ames-test strains and reduced NaN₃ induced reversions. There was a significant decrease in the vitality (IC₅₀=0.18 mM) of cancer cells.

Treatment with the modified sodium silicate also resulted in increased antioxidant response as seen from fold increases in activities of antioxidant enzymes SOD and CAT. The levels of impotent cellular antioxidant molecule, GSH, was also found to increase in a dose dependent manner. This was compounded with a decrease in MDA, an oxidative stress biomarker.

Based upon the above results, it appeasers that modified sodium silicate has the potential to decrease initial events in carcinogenesis by modulating redox mediated events, enhancing antioxidant response, promoting apoptosis and decreasing DNA mutations.

### Anti-Viral Activity:

It has been demonstrated that modified sodium silicate has the following anti-viral effects, and in particular, anti-retroviral effects:
modified sodium silicate increased nitric oxide dependent anti-viral effects at all concentrations tested;
modified sodium silicate inhibited enzymes important in viral assembly, metabolism and replication;
modified sodium silicate caused changes in the viral carbohydrate composition and metabolism; and
modified sodium silicate inhibited the activity of the enzyme responsible for transcribing RNA to DNA in the virus and the effects were dose dependent.

### Materials and Methods:

### Nitric Oxide Production

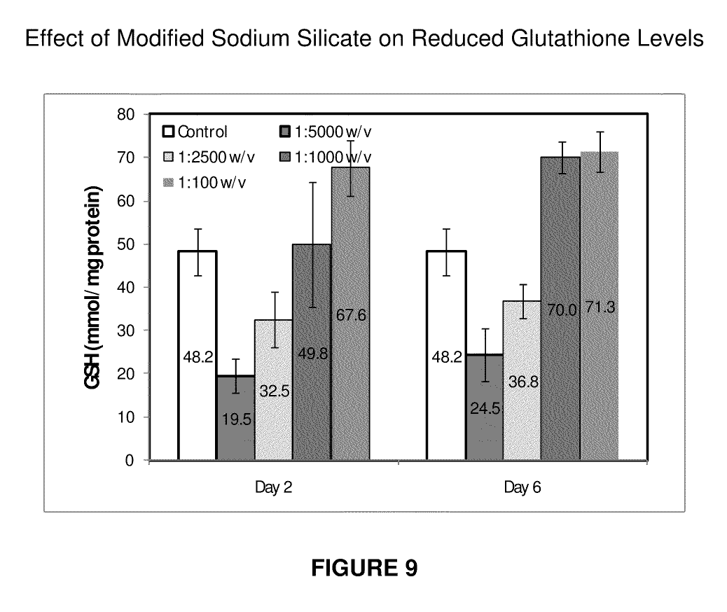

Nitric oxide production was measured using a modified Griess assay for the detection of total nitrites. Briefly, 100 μl of whole cell extract was transferred to a microplate followed by addition of 100 μl of vanadium chloride (0.08 g/10 mL 0.1 M HCl) and 100 μl Griess reagent. Alternatively, 50 μl sulfanilamide and 50 μl N-(1-Naphthyl)ethylenediamine dihydrochloride (NEDD) can be substituted for Griess reagent in the reaction. The microplate was incubated for 30 minutes at 37° C. and absorbance was measured at 540 nm using the Bioteck EL 808 (Houston, Tex.). The concentration of nitric oxide was determined by calculating the % change based on a linear standard curve equation: [Conc (umol/L)=(A540−0.0344)/0.0057)]. The experiment demonstrating NO production is discussed further below in the examples and the results for Days 2 and 6 are shown in FIG. 9.

### Reverse Transcriptase (RT) Assay

The effect of different concentrations of modified sodium silicate on reverse transcription was tested using a non-radioactive HIV-RT colorimetric ELISA kit from Roche Diagnostics, Germany. The protocol outlined in the kit was followed using 2 ng of enzyme in a well and incubating the reaction for 2 hours at 37° C.

### Glycohydrolase Enzyme Assays

Glycohydrolase enzymes are found in the eukaryotic host cell's Golgi apparatus and are responsible for glycosylation of proteins. Inhibition of the glycohydrolase enzymes has been found to decrease the infectivity of the HIV virion, as the HIV envelope proteins are highly glycosylated during the life cycle of the virus. α-Glucosidase has been found to be partly responsible for the glycosylation of HIV gp120.

To measure the inhibition of the glycohydrolase enzymes; α-glucosidase (Sigma, Mo., USA), β-glucosidase (Sigma, Mo., USA) and β-glucuronidase (Roche Diagnostics, Germany) were used with their corresponding substrates ρ-nitrophenyl-α-d-glucopyranose, ρ-nitrophenyl-β-d-glucopyranose and ρ-nitrophenyl-β-d-glucuronide (Sigma, Mo., USA) in a colorimetric 96-well microtiter plate-based assay, determining the amount of ρ-nitrophenol released. The method described by Collins et al., 1997 and Collins et al., 1997 was followed with modifications. Briefly, substrates and enzymes were dissolved in their appropriate 50 mM buffers (2-morpholinoethanesulphonic acid monohydrate (Mes)-NaOH (Sigma, Mo., USA), pH 6.5, for α-glucosidase and β-glucuronidase and sodium acetate, pH 5.6, for β-glucosidase). The final assay volume was 200 μl and contained 2 mM substrate, 0.25 μg enzyme, and the crude extract at 0.2 mg/ml. The reaction was allowed to proceed for 15 minutes at 25° C. before termination with 60 μl 2 M glycine-NaOH, pH 10, and measurement of absorbance at 412 nm.

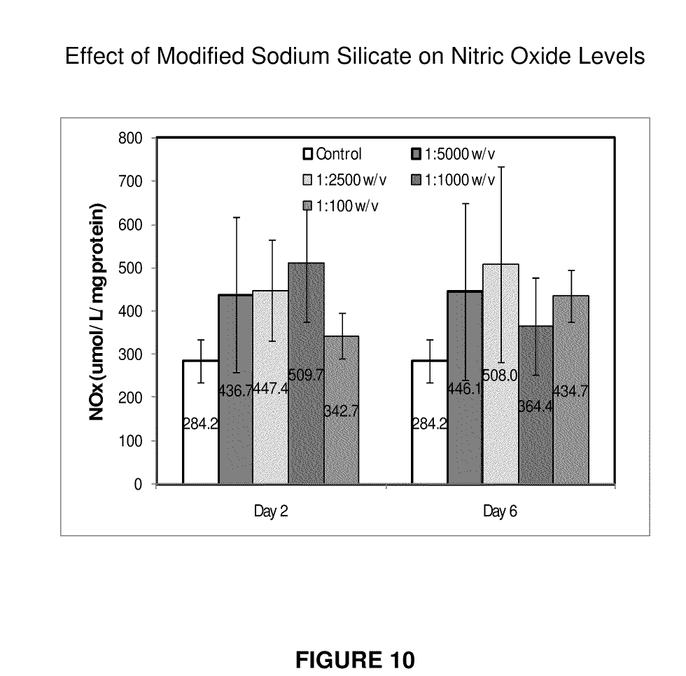

The experiment demonstrating inhibition of HIV envelope protein glycosylation for various concentrations of modified sodium silicate is discussed further below in the examples and the results are shown in FIG. 10.

### Protease (PR) Assay

The procedure for the fluorometric detection of HIV-PR activity was carried out as described by Au et al., 2000 and Au et al., 2000 using HIV-II PR. The HIV-II PR was obtained from the NIH AIDS Research and Reference Reagent Program, NIAID, NIH, MD, USA in the form of 100 μg in 100 mM sodium phosphate (pH 8), 50 mM NaCl buffer. A fluorescence resonance energy transfer (FRET) assay using the fluorogenic substrate, DABCYL-γ-Abu-Ser-Gln-Asn-Tyr-Pro-Ilee-Val-Gln-EDANS (Bachem, Switzerland), was used to assay HIV-PR. Substrate (10 μM) was added to a 200 μl reaction sample that included 100 nM HIV-II PR, reaction buffer (0.1 M sodium acetate, 1 M NaCl, 1 mM EDTA, 1 mM DTT, 10% DMSO, 1 mg/ml BSA, pH 4.7) and modified sodium silicate at different concentrations. This was incubated at 37° C. for 2 hours. The fluorescence intensity is indicative of protease activity and was measured at an excitation wavelength of 355 nm and emission wavelength of 460 nm.

### Anthrone Assay

40 μl water (blank), standard (0.05, 0.15, 0.2, 0.25, 0.3 and 0.4), or sample was added to each well of a 96-well microtiter plate. To the wells, 0.1 ml anthrone solution (freshly prepared) was added. The plates were mixed well and incubated at 92° C. for 3 minutes in a non-shaking water bath. Plates were then transferred to a non-shaking water bath at RT for 5 minutes to stop the reaction and absorbance was read at 600 nm.

### Ferric-Orcinol Assay (Bial's Test)

To 200 μl of sample, 200 μl of 10% TCA was added and heated at 100° C. for 15 minutes. Tubes were rapidly cooled at 25° C. and 1.2 ml of the following reagent: (1.15% w/v ferric ammonium sulfate and 0.2% w/v orcinol in 9.6M HCL) was added and mixed thoroughly. Samples were again heated at 100° C. for 20 minutes and cooled to room temperature. Absorbance of the blue-green color was measured at 660 nm.

### Sialic Acid Assay Method

To a sample, 0.1 ml 0.04M periodic acid was added and thoroughly mixed and incubated in an ice bath for 20 minutes. 1.25 ml resorcinol reagent was added mixed and placed in an ice bath for 5 minutes. The solution was heated at 100° C. for 15 minutes and cooled in tap water after which 1.25 ml of tert-butyl alcohol was added and mixed vigorously to give a single phase solution. The tubes were then placed in a 37° C. water bath for 3 minutes to stabilize the color and absorbance read at 630 nm.

### Uronic Acid Assay

To 40 μl sample containing 200 μl concentrated sulfuric acid (96%) w/w containing 120 mM sodium tetraborate was carefully added in a microplate and mixed. The plates were incubated at 80° C. for 1 h and cooled. 40 μl m-hydroxydiphenyl reagent (100 μl of m-hydroxydiphenyl in dimethyl sulfoxide, 100 mg/ml mixed with 4.9 ml 80% sulfuric acid just before use) was added to each sample and mixed. After incubating for 15 min at room temperature, absorbance was read at 540 nm.

### Heptose Assay

To 50 μl of sample, 50 μl 0.5 N H₂SO₄ was added and vortexed.

The mixture was placed in a 100° C. water bath for 8 min and cooled to room temperature. To this 50 μl, H₅IO₆ was added and mixed. This mixture was incubated for 10 min at room temperature and 100 μl arsenite reagent was added and mixed until the yellowish color disappeared. To this 200 μl, thiobarbituric acid reagent was added and incubated at 100° C. for 10 min and cooled. 1.5 ml butanol reagent was added and the solution was vortexed. 125 μl of DMSO was then added and absorbance was read at 550 nm.

### Anti-retroviral Effects

Modified sodium silicate was evaluated for its in vitro anti-retroviral effects. Assays for inhibition of HIV-II reverse transcriptase (RT), HIV-II protease (PR) and glucohydrolase [glucuronidase (GH-1) and glucosidase (GH-2)], both of which are important for viral replication, coat assembly and virulence, respectively, were performed using standard kits. The effects on nitric oxide (NO)_dependent antiviral activities were measured in neutrophils using standard assays.

The modified sodium silicate significantly decreased HIV-Rt activity in a dose dependent manner (ED₅₀=20.4 mM). HIV-PR activity decreased (EC₅₀=14.64 mM) with increasing product concentration.

The modified sodium silicate also decreased the HIV-II virulence by inhibiting the GH-1 (IC₅₀=0.06 mM) and GH-2 (IC₅₀=14.6 mM) activity, which decreased protein glucosylation and glucuronylation. Higher BO was detected in neutrophil medium, suggesting an increase in NO mediated antiviral activity.

The modified sodium silicate was quantified by the ammonium molybdate assay. The silicon-hydrate complex formed was quantified at 450 nm. NMR and IR spectroscopy were used for structural determination. In the case of NMR, a Magic Angle Spinning (MAS) technique of high resolution as used in the ¹H₁ nuclei. For the IR study, a Fourier Transform methodology was used for spectral analysis.

The anti-glucohydrolase effects were evaluated at 412 nm using p-nitrophenol substrate. L. terrestris was cultured using standard culture techniques. Nitric oxide formation by neutrophils was measured as total nitrates (NOₓ) using the Friess reaction. Protein was measured using the Bradford assay.

The effect of different concentrations of the modified sodium silicate on reverse transcription was tested using a non-radioactive HIV-RT colorimetric ELISA kit from Roche Diagnostics, Germany. Protease activity was measured by a fluorometric assay using a HIV-1 SensoLyte® HIV Protease Fluorimetric Assay Kit (anaSpec USA).

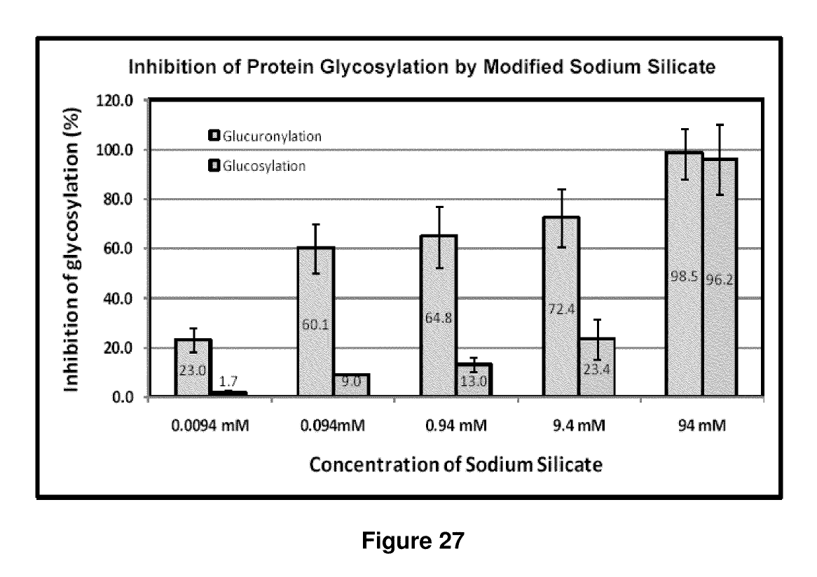

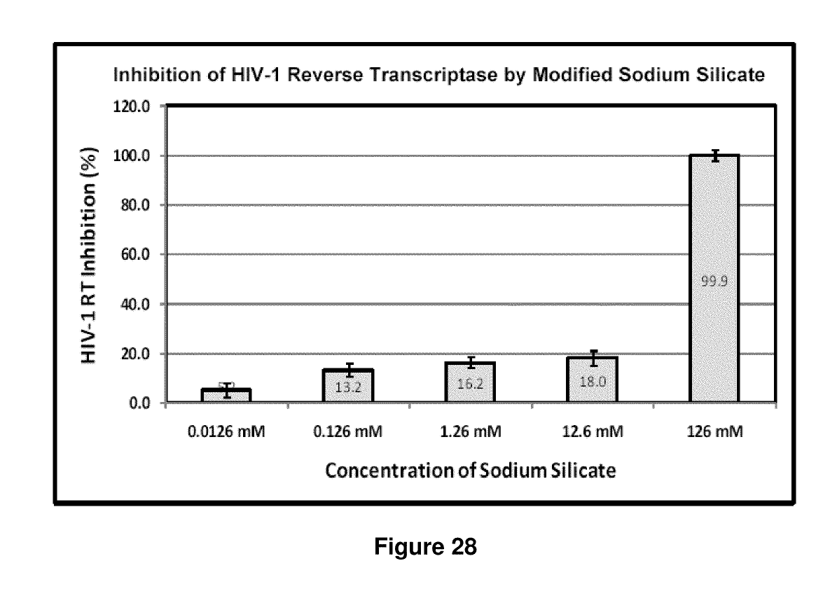

The results of these assays are shown in FIGS. 27-30.

### EXAMPLES

### Example 1

### Production of Modified Sodium Silicate

The following example describes a representative method for making the modified sodium silicate of the present application,

To make 10 gallons of the modified sodium silicate at 1.25 specific gravity, the following ingredients were used:

| | | |
|---|---|---|
| Initial amount of silicon rock to start the reaction | 46.7 | pounds |
| Water at 150° F. | 5.5 | gallons |
| Sodium hydroxide at 50% | 2.05 | gallons |

Reaction process for the first batch:

First, the silicon rock was introduced into a 30 gallon reactor. Note: after the initial reaction, the amount of silicon rock that will be needed to start the reaction for a subsequent second batch and every other thereafter will be only 7.85 pounds.

Second, approximately half of the total volume of the heated water was added to the reactor.

Third, sodium hydroxide was added, while continuing to add the water.

Fourth, the remaining water was added.

Fifth, after all components are added to the reactor, an exothermic reaction occurred for 4 to 6 hours (for the first time batch) (Note: for subsequent batches, the reaction time required will be less). To determine if the reaction is finished, there should be very little reaction bubbles on top of the liquid in the reactor; instead, there are mostly large bubbles and no vapor coming out.

Then, the reaction product was emptied into a tank. This product had very high (like molasses) inconsistency with a temperature of approximately 195 to 200° F. After the reaction product was removed from the reactor, water was sprayed over the remaining silicon rocks in the reactor to wash the rocks, and this washed liquid was emptied into the same tank.

The reaction product was allowed to cool at ambient room temperature. After the reaction product had completely cooled, water was added (while continuously mixing) until a specific gravity (weight in grams of liquid divided by volume in milliliters) of 1.25 was reached. For equal specific gravity, the liquid was allowed to settle for approximately 3 to 4 hours to drop all sediment (black inert material). The liquid was then pumped through a fine filter (1 to 3 microns) and into a storage tank. At this point, the resultant liquid product is ready for packaging into containers and ready to be used. It is non-toxic and not corrosive.

The product obtained was an aqueous solution of Na₈₂Si_{4.4}H_{9.7}O_{17.6} with the following properties: a specific density of 1.25+/−, a boiling point of 210° F., a freezing point of 32° F., a pH of 13.9+/−, and solubility in water: miscible 100%.

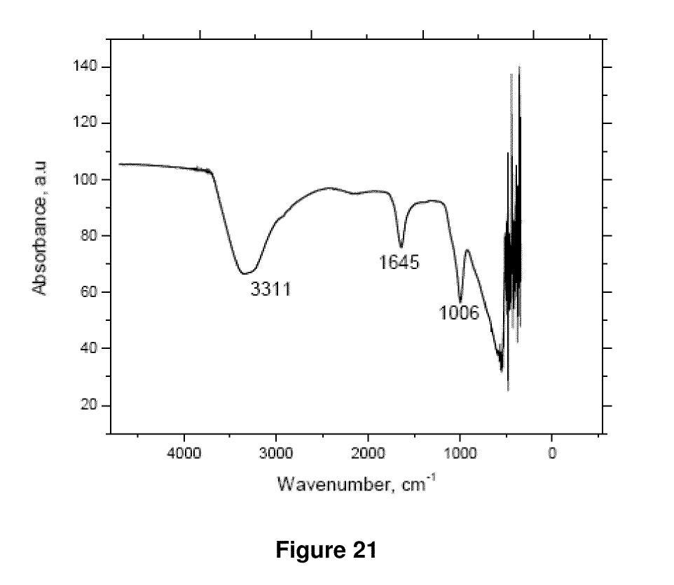

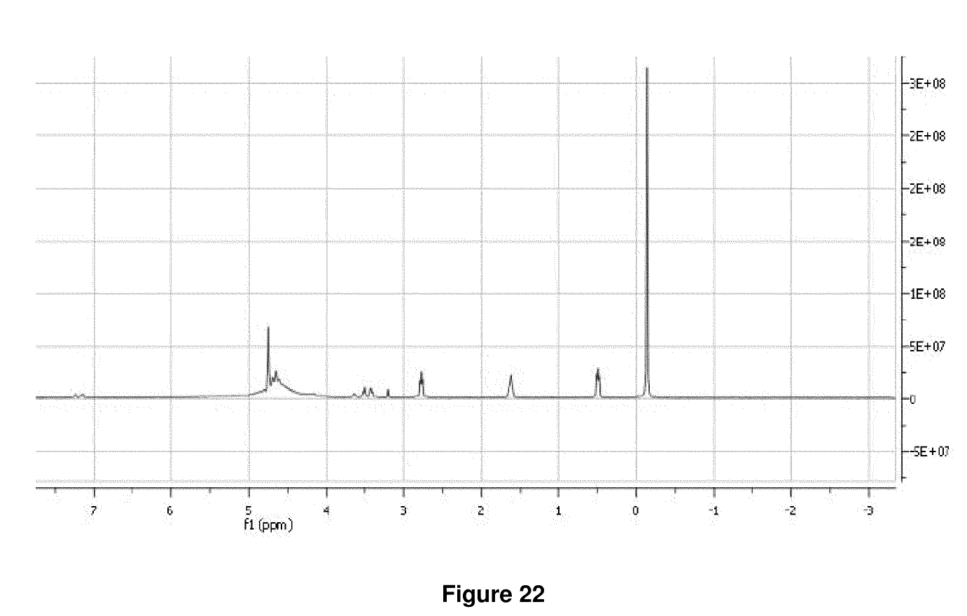

Nuclear magnetic resonance (NMR) and infrared spectroscopy (IR) were performed on the on the resultant product. FIGS. 21 and 22 show the results of NMR and IR studies, respectively. For NMR, an Magic Angle Spinning (MAS) technique of high resolution was used in the ¹H nuclei; meanwhile, for the IR study, Fourier Transform (FT) method was performed to analyze the spectra.

The target solution was analyzed by an IR spectrometer operated in transition mode. FIG. 21 shows three well-resolved vibration signals, the first one at 3311 cm⁻¹ normally attributed to the water molecule in the sample according to discrimination rules followed. The next signals are at 1645 and 1006 cm⁻¹ and are not strain-forward and can be assigned because they can be due to the hydroxyl compounds based on Na and Si or due to the vibration of the Na and Si ions, respectively. These signals can be due to the free OH group. The results shown in FIG. 21 suggest that the only functional groups present in this sample are water and OH groups.

More analysis was carried out using ¹H MAS-NMR to complement the above results. By ¹H MAS-NMR spectroscopy it was possible to confirm that, in the sample, there are no functional groups other than OH groups. FIG. 22 shows the main resonance of the proton at ˜0 ppm is attributed to water. On the other hand, the tiny peaks observed upwards of 7 ppm can be related to the resonance of the hydroxyl group attached to the inorganic components, Na and/or Si.

²³Na and ²⁹Si NMR experiments can be done to further study the nature and number of substitutes present in the Na and Si shell. Also, the crystalline structure of the sample can be investigated using X-ray diffraction (XRD).

### Anti-Cancer Effect

### Example 2

### Effect of Modified Sodium Silicate on Survival of Colon Cancer Cell Line HT-29

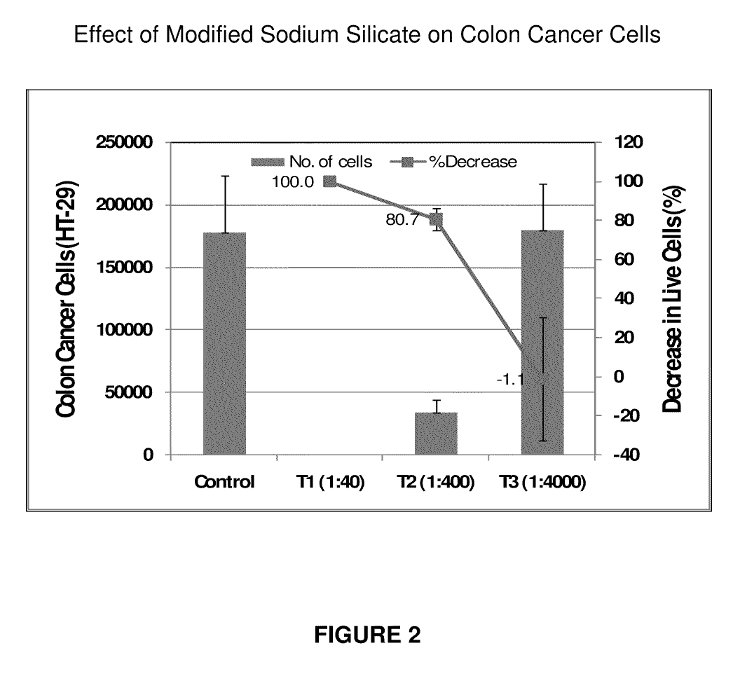

Modified sodium silicate was diluted in distilled deionized (DDI) water 1:40; 1:400 and 1:4000 times. This diluted product was added to cell culture media seeded with colon cancer cells (HT-29) in a 16 well plate and allowed to grow overnight under the conditions described above. The following day, the number of surviving cancer cells were counted and recorded. Compared to the control, it was observed that cells treated with modified sodium silicate at 1:40 diluted completely killed all the cancer cells (100% lethality). At 1:400 dilution of modified sodium silicate only 20% of the cancer cells survived (80% lethality) and at 1:4000 dilution it was not effective in killing colon cancer cells, which suggests that the EC₅₀ (concentration at which 50% of the cells were killed) is approximately 1:250 dilution (FIG. 2).

### Example 3

### Effect of Modified Sodium Silicate on Attachment of Colon Cancer Cell Line HT-29 to Surfaces

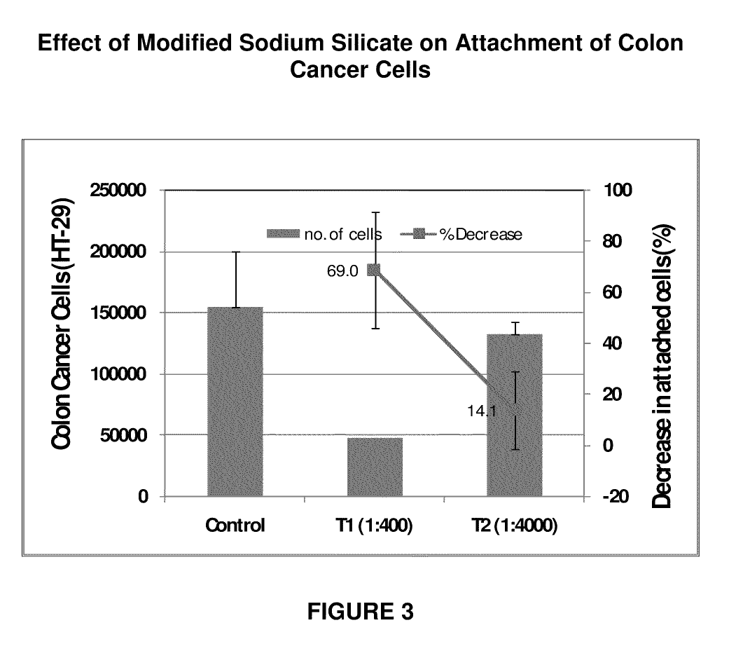

Attachment of cancer cells is a fundamental process involved in the establishment of cancer, its spreading and eventual metastasis. All of these processes are required for carcinogenesis and eventual morbidity and mortality that results from it. Modified sodium silicate was diluted in distilled deionized (DDI) water 1:40; 1:400 and 1:4000 times. This diluted product was added to cell culture media seeded with colon cancer cells (HT-29) in a 16 well plate and allowed to grow overnight under the conditions described above. The following day, plates were washed to remove detached cells. The cells that were still attached were trypsinized and enumerated under the microscope using a hemocytometer. Compared to the control, it was observed that cells treated with modified sodium silicate at 1:40 dilution it completely prevented attachment of all the cancer cells (100% effective) (FIG. 3). At 1:400 dilution of modified sodium silicate only 31% of the cancer cells attached (69% effective) and at 1:4000 dilution only 86% of the cancer cells attached (14% effective), which suggests that the EC₅₀ (concentration at which 50% of the cells were killed) is approximately 1:290 dilution.

### Example 4

### Effect of Modified Sodium Silicate Against Various Types of Mutations Induced by Sodium Azide in the Ames Test for Mutations

Modified sodium silicate was tested at various dilutions for its ability to inhibit various types of mutations induced in the salmonella tester strains in response to sodium azide. In all the strains except in TA102, the product was able to reduce mutations by 80-100% at 1:40 times dilution. At 1:400 dilution, it was able to prevent 97-100% missesense (TA1535) deletion-frameshift mutations (TA1537) and 17% of missense mutations in TA100. At the highest dilution (1:4000), the product was able to prevent 20% missense mutations in TA100; 60% missense mutations in TA1535 and 86% deletion frame-shift mutations in TA1537. Modified sodium silicate may be inhibiting the binding of the mutagen to DNA by blocking the mutagen binding sites.

NaN₃ is known to cause a mismatch in DNA replication by substituting for natural thiol groups from cysteine and methionine and then binding to the DNA. It is also likely that modified sodium silicate could possibly inhibit the mutation induced by NaN₃ by preventing the binding of 13-azidoalanine to the DNA bases by blocking its DNA binding site and by maintaining the activity of the enzyme O-acetyl serine thiol lyase, which has DNA protective functions. A consequence of DNA mutation in response to NaN₃ is the induction of recA dependent SOS response which identifies the mutated base and removes it, creating an “empty” base.

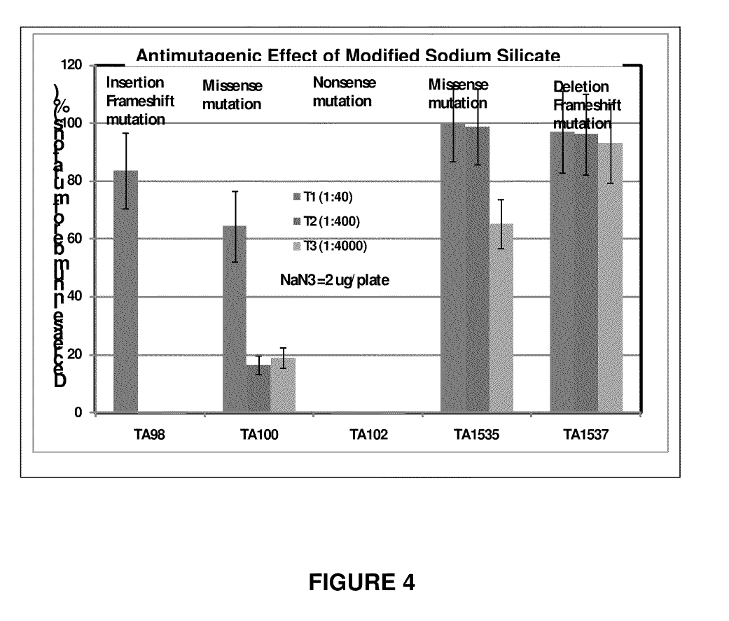

As a more effective redox modulator, it appears that the mechanism for inhibition of mutagen induced SOS response by modified sodium silicate is by the suppression of inactivation of the LexA repressor by the RecA protease, suppression of the transcription of the recA gene, and suppression of RecA protein synthesis as well as induction of adaptive/inducible repair systems consisting of several proteins that recognize very specific modified bases. See the results in FIG. 4.

### Example 5

### Apoptotic Effect of Modified Sodium Silicate at Various Concentrations as Measured by Fragmented DNA

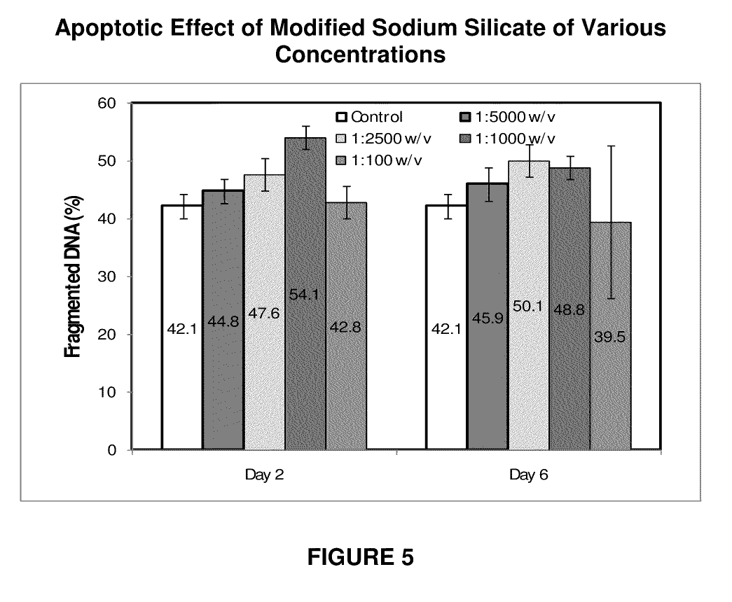

Induction apoptosis or programmed cell death is an effective mechanism by which cancer development and progression can be controlled. One of the hallmarks of apoptosis is the formation of fragmented DNA due to the induction of apoptotic processes such as chromatin condensation. Modified sodium silicate was diluted in DDI water 1:100; 1:1000, 1:2500 and 1:5000 times and sustained apoptotic effect of modified sodium silicate was measured for 6 days. It was observed that both at day 2 and day 6 the modified sodium silicate exhibited a classic response curve. In general at all dilutions the modified silicate was more effective on day 2 compared to day 6, however, even at day 6 the product retained significant activity. On both day 2 and day 6, the apoptotic activity increased with increase in concentration of modified sodium silicate (FIG. 5), with the product being most effective at 1:1000 dilution. At 1:100 dilution, the amount of fragmented DNA decreased, which is a commonly observed effect since beyond a critical concentration modified sodium silicate might be promoting death of cancer cells independent of induction of apoptosis.

### Example 6

### Effect of Modified Sodium Silicate at Various Concentrations on Free Radical Formation

The effect of modified sodium silicate on the redox homeostasis was investigated by measuring the amount of free radical induced formation of malondialdehyde (MDA). Oxidation of biological molecules by reactive oxygen species (ROS) results in initiation of tumorigenic and carcinogenic processes. MDA is a secondary oxidation product of lipids and serves as a good marker for oxidation and cell injury. For constant removal of ROS from the system, it is essential for the cells to replenish cellular antioxidant pools either by reducing oxidized antioxidants or by inducing synthesis of cellular antioxidants and antioxidant enzymes. In an actively metabolizing tissue, this ROS is quickly removed with the help of several cellular antioxidants and cellular antioxidant enzymes such as GSH, SOD and CAT.

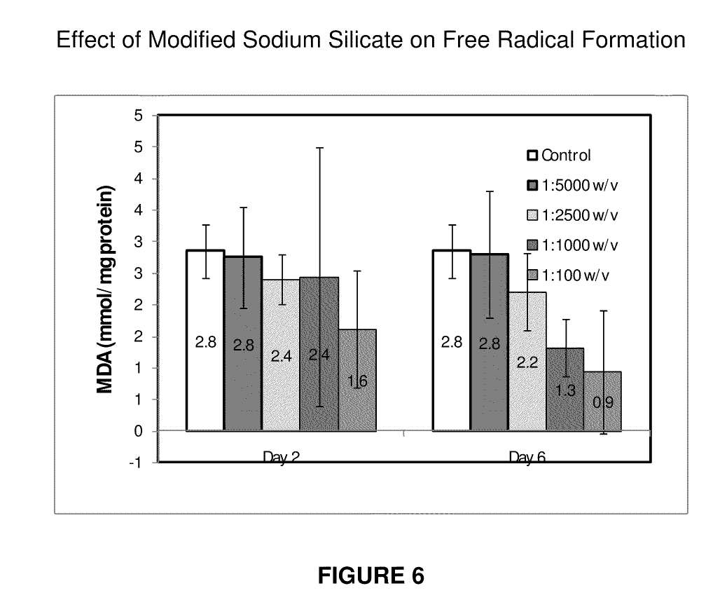

Modified sodium silicate was diluted in DDI water 1:100; 1:1000; 1:2500 and 1:5000 times and the sustained effect of modified sodium silicate was diluted on reducing free radical induced oxidation for 6 days was evaluated. It was observed that both at day 2 and day 6 the compound exhibited a dose response curve. The compound was equally effective on day 2 and day 6. On both day 2 and day 6, the MDA values decreased linearly with an increase in concentration of modified sodium silicate (FIG. 6), with the product being most effective at 1:100 dilution.

### Example 7

### Effect of Modified Sodium Silicate at Various Concentrations on SOD Activity

SOD is responsible for converting superoxide to hydrogen peroxide, which is then degraded by CAT. SOD exists in combination with a transition metal that corresponds to the transition metal that catalyzes the reaction that forms the ROS; for example, Mn SOD, CuZn SOD, Fe SOD, and Ni SOD. CuZn SOD is restrained in the presence of hydrogen peroxide. It is an important antioxidant defense in nearly all cells exposed to oxygen and high activity of SOD is linked to lower incidences of several forms of cancer.

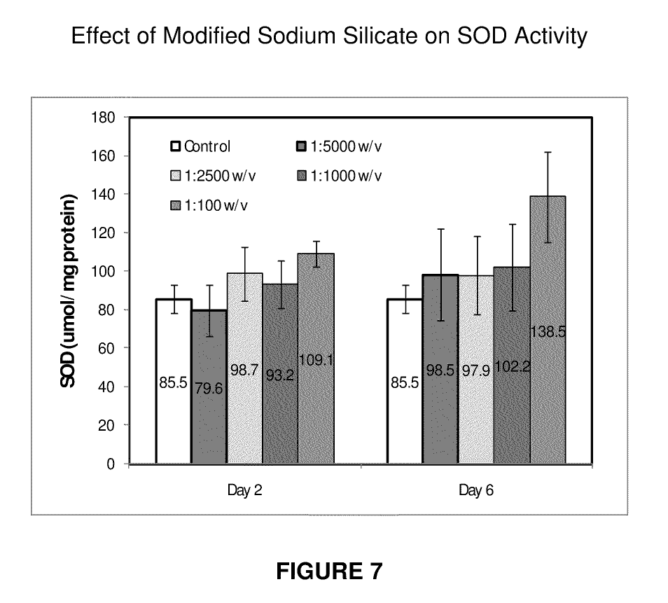

The ability of various concentrations of modified sodium silicate to increase the expression and activity of an important antioxidant enzyme, SOD, was investigated. Modified sodium silicate was diluted in DDI water 1:100; 1:1000, 1:2500 and 1:5000 times and the sustained effect of modified sodium silicate on SOD activity for 6 days was measured. It was observed that both at day 2 and day 6 the modified sodium silicate exhibited a dose response curve. The effectiveness of the treatment increased with the time and on day 6 the SOD activity was higher than compared to day 3 levels. On both day 2 and day 6, SOD activity in the cells increased linearly with an increase in concentration of modified sodium silicate (FIG. 7) with the product being most effective at 1:100 dilution. At this dilution the increase in SOD activity was 109% on day 2 and 138% on day 6.

### Example 8

### Effect of v at Various Concentrations on Catalase Activity

CAT is responsible for converting the hydrogen peroxide ROS to water and oxygen; it is a very efficient enzyme because it quenches hydrogen peroxide regardless of hydrogen peroxide concentration. Deficiency of CAT is observed in several types of cancer and increased activity is related to lower vigor in cancer cells.

The ability of various concentrations of modified sodium silicate to increase the expression and activity of CAT was investigated. Modified sodium silicate was diluted in DDI water 1:100; 1:1000, 1:2500 and 1:5000 times and the sustained effect of modified sodium silicate on CAT activity for 6 days was measured. It was observed that both on day 2 and day 6 at 1:100; 1:1000 dilutions an increase in CAT activity occurred, with higher levels observed at 1:100 dilution. On day 6 even at 1:2500 dilution increased activity in CAT was seen. (FIG. 7). The decrease in CAT activity with other dilutions is perhaps because of decreased hydrogen peroxide production due to lower ROS production and SOD activity.

### Example 9

### Effect of Modified Sodium Silicate at Various Concentrations on Reduced Glutathione Levels

Secondary antioxidant defense molecules protect against oxidation by quenching ROS. Secondary defense encompasses small antioxidant molecules, including glutathione, vitamins E and C, coenzyme Q, uric acid, and carotenoids (12). Small antioxidant molecules are found in the diet. Glutathione (GSH) has been described for a long time just as a defensive reagent against the action of toxic xenobiotics (drugs, pollutants, carcinogens). As a prototype antioxidant, it has been involved in cell protection from the noxious effect of excess oxidant stress, both directly and as a cofactor of glutathione peroxidases. In addition, it has long been known that GSH is capable of forming disulfide bonds with cysteine residues of proteins, and the relevance of this mechanism (“S-glutathionylation”) in regulation of protein function. Lower levels or deficiency is responsible for several types of cancer.

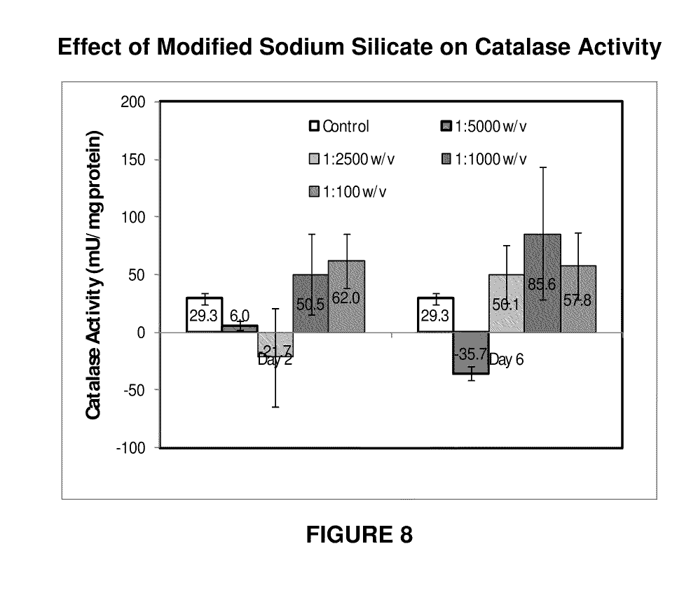

The ability of various concentrations of modified sodium silicate to increase the expression and activity of an important antioxidant molecule, GSH was investigated. Modified sodium silicate was diluted in DDI water 1:100; 1:1000, 1:2500 and 1:5000 times and the sustained effect of modified sodium silicate on GSH levels was measured for 6 days. It was observed that both on day 2 and day 6 at 1:100; 1:1000 dilutions increase in CAT activity was observed, with higher levels observed at 1:100 dilution (FIG. 8). On day 2, modified sodium silicate at 1:100 dilution increased the concentration of GSH by 67 mmol whereas, an increase in GSH levels to 70 mmol was seen at 1:100; 1:1000 by day 6. The decrease in GSH levels similar to CAT levels with other dilutions is perhaps because decreased hydrogen peroxide production due to lower ROS production and SOD activity.

### Anti-Viral Effect

### Example 10

### Effect of Various Concentrations of Modified Sodium Silicate on Nitric Oxide Levels as Measured by Total Nitrates

The immune system uses nitric oxide for fighting viral, bacterial and parasitic infections. Nitric oxide is a reactive nitrogen species produced from the conversion of L-arginine to citrulline via nitric oxide synthase (NOS). Nitric oxide is highly reactive due to its reaction with other ROS to form peroxynitrite; it is also a vasodilator and therefore is involved in the control of blood pressure via the stimulation of guanylate cyclase. Three forms of NOS exist, including neuronal NOS-I, endothelial NOS-III, and NOS-II. NOS-I and NOS-III are constitutive, while NOS-II is inducible in macrophages. It is physiologically a very important molecule and plays important role in adhesion molecule/chamomile expression, leukocyte recruitment for fighting off viral infections. Being a neurotransmitter gas, it plays an important role in long-term potentiating and in synaptic plasticity, both of which are important for memory development. Additionally, it is also called an endothelium derived relaxation factor (EDRF) and plays an important role in blood pressure reduction and reproductive health. NO also decreases proliferation of tumors and is also associated with learning, memory, sleeping, feeling pain, and, probably, depression.

To demonstrate the effect modified sodium silicate on NO production, modified sodium silicate was diluted in DDI water 1:100; 1:1000, 1:2500 and 1:5000 times and measured the sustained effect of modified sodium silicate on NO levels for 6 days. Modified sodium silicate at all concentrations, increased the NO levels for the duration of the treatment. On day 2, the levels of NO were found to be: 437 umol, 447 umol, 509 mmol and 342 mmol at 1:5000, 1:2500, 1:1000, 1:100 dilutions, respectively (FIG. 10). Whereas on day 6, these levels changed to 446 umol, 508 umol, 364 umol, 434 umol respectively, suggesting a sustained effect of modified sodium silicate on NO levels.

### Example 11

### Effect of Various Concentrations of Modified Sodium Silicate on HIV Envelope Protein Glucosylation and on HIV Envelope Protein Glucuronylation

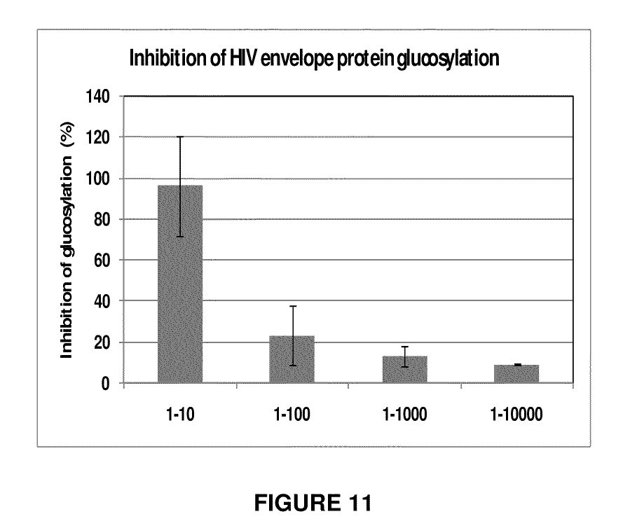

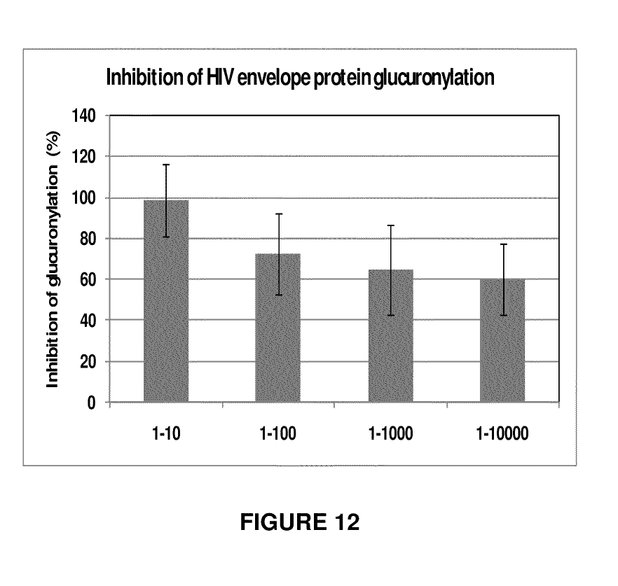

The HIV virus evades the immune system and attaches to cells using surface sugars. If this process can be inhibited, initial infection can be prevented. Glycohydrolase enzymes are found in the eukaryotic host cell's Golgi apparatus and are responsible for glycosylation of proteins. Inhibition of the glycohydrolase enzymes has been found to decrease the infectivity of the HIV virion, as the HIV envelope proteins are highly glycosylated during the life cycle of the virus. Alpha-glucosidase has been found to be partly responsible for the glycosylation of HIV gp120. Inhibitors of glycosylation could have potential therapeutic use by interfering with viral maturation, attachment and evasion. Modified sodium silicate was diluted in DDI water 1:10; 1:100; 1:1000, and 1:10000 times and measured the effect of modified sodium silicate on viral envelop glucosylation and glucuronylation was measured (FIG. 11 and FIG. 12, respectively). It was observed that modified sodium silicate reduced glucosylation of the viral envelop protein in a dose dependent manner (FIG. 11). At 1:10 dilution, the product completely inhibited the activity if glucohydrolase, with further dilution resulting in a linear decrease in inhibition. The EC5₀ for inhibition of glucosylation was calculated to be 1:40 dilution. Modified sodium silicate also inhibited the glucuronylation of viral protein in a similar dose dependent manner. At 1:10 dilution, the modified sodium silicate caused 98% inhibition in the enzyme activity. Further dilution resulted in a linear decrease in the enzyme activity. At the lowest concentration, modified sodium silicate caused compound still retained glucuronylation inhibition activity of 60%, suggesting the EC5₀ for this assay was much lower than the concentrations tested in this investigation (FIG. 12).

### Example 12

### Effect of Various Concentrations of Modified Sodium Silicate on ribose concentration, heptose concentration, sialic concentration, and Uronic Acid Concentration

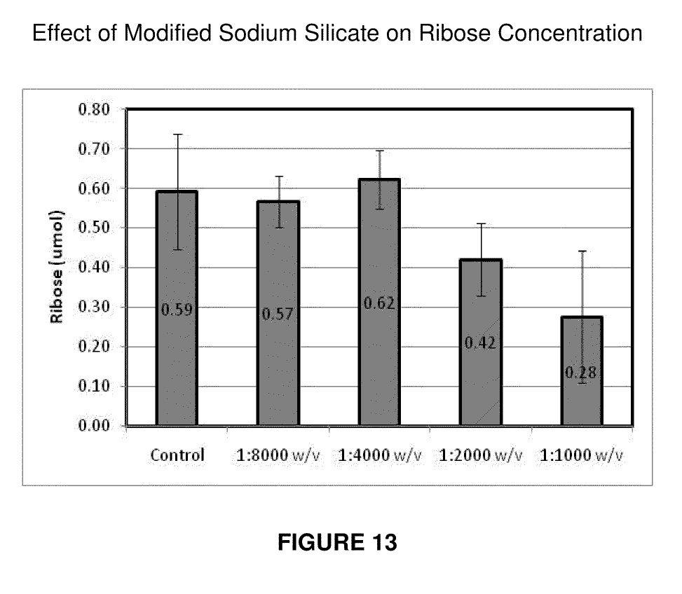

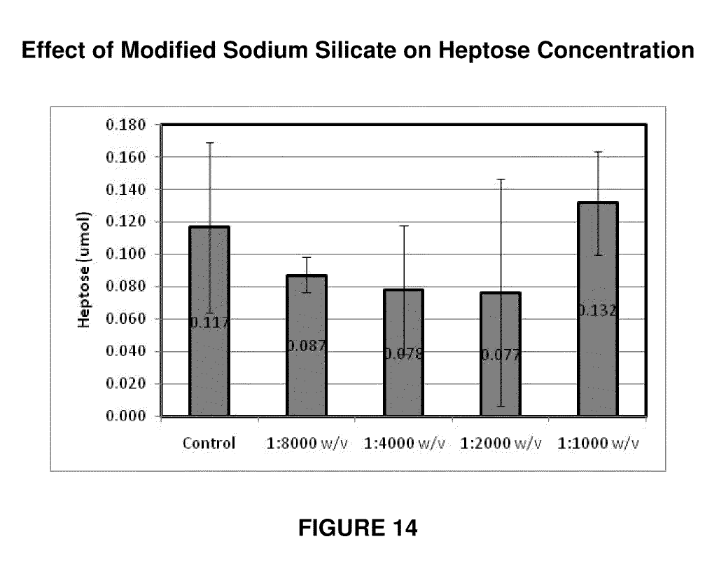

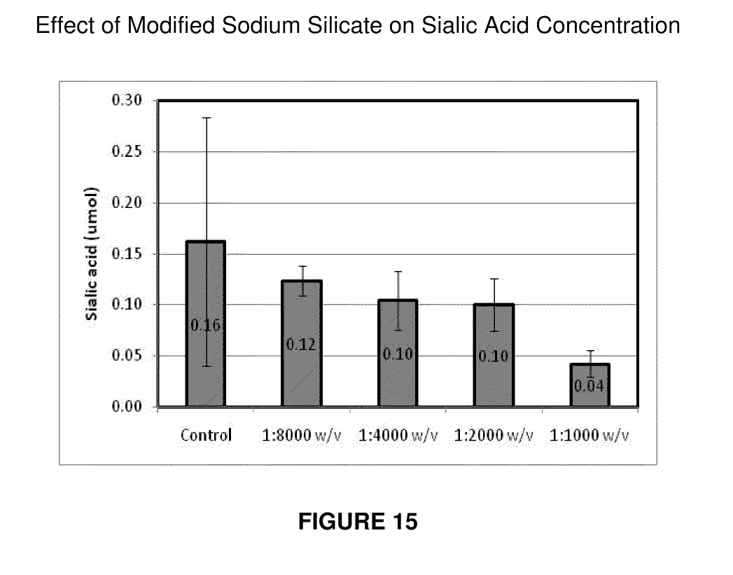

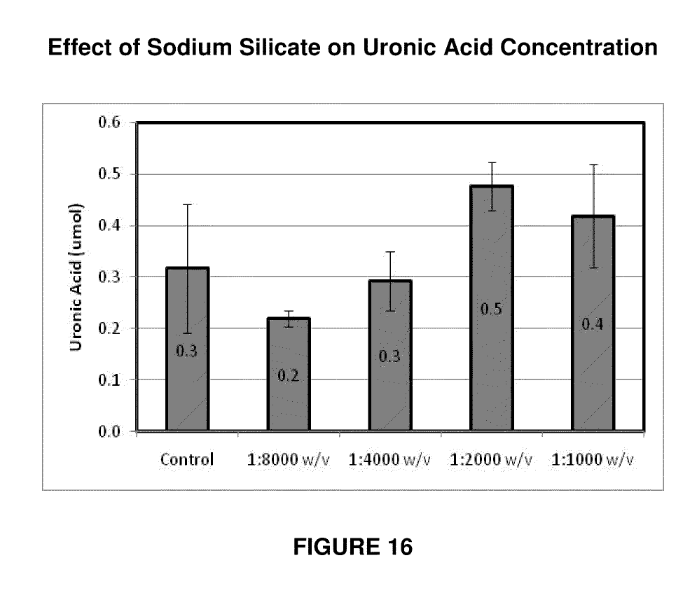

In addition, experiments confirmed that the inhibition in viral glycoprotein post translational modification resulted in an actual changes in the viral carbohydrate composition. Using the assays described in the previous section, the concentration of various types of sugars, including ribose, heptose, sialic acid and uronic acid, levels upon treatment with different concentrations of modified sodium silicate. This is an important parameter to evaluate, as different sugars determine the conformation and specificity of interaction with receptor systems. This conformation and specificity can be altered by changing sugar concentration, which renders the protein not suitable for receptor interaction. This inability to bind to a cell surface receptor prevents attachment to cells for invasion and makes them susceptible for clearance by the immune system. It must be noted that the objective here was to just monitor changes in the sugar composition and not necessarily increase or decrease, as just a change in concentration is enough to induce non-specificity. The results indicated a change in the levels of ribose (FIG. 13), heptose (FIG. 14), sialic acid (FIG. 15) and uronic acid (FIG. 16) upon treatment with different concentrations of modified sodium silicate.

### Example 13

### Effect of Various Concentrations of Modified Sodium Silicate on HIV-1 Reverse Transcriptase Activity

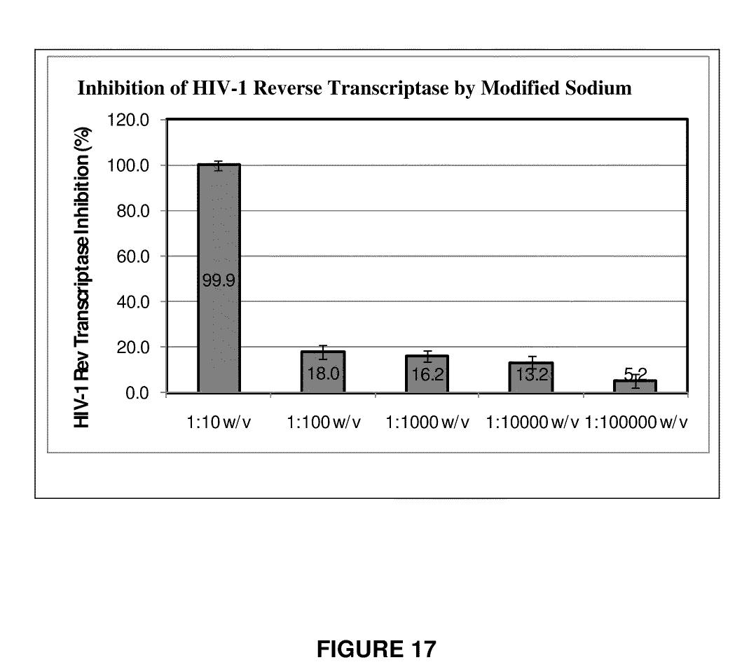

Before retroviral infections establish themselves, their nucleic material of RNA needs to be first transcribed into a double stranded DNA by a viral enzyme known as viral reverse transcriptase (RT). Once RT synthesizes DNA, it is ligated into the host genome where it replicates along with the host DNA. When conditions are favorable, the viral DNA is transcribed and translated to make all proteins necessary for the viral assembly. However, without reverse transcriptase, the viral genome couldn't become incorporated into the host cell, and couldn't reproduce. This important step had made anti-RT therapy an important target for developing antiviral drugs for a number of retroviral infections. Modified sodium silicate was diluted in DDI water 1:10; 1:100; 1:1000, 1:10000 and 1:100000s times and measured the effect of modified sodium silicate on HIV-1 reverse transcriptase activity was measured. The results showed that at 1:10 dilution, modified sodium silicate completely inhibited the activity of the enzyme resulting in the synthesis of very low levels of HIV-1 DNA (FIG. 16). Upon further dilution, the inhibition activity decreased significantly and at 1:100; 1:1000, 1:10000 the inhibition ranged between 13-18% respectively and 1:100000 the inhibition activity further decreased to 5% compared to the control (FIG. 17).

### Example 14

### Effect of Various Concentrations of Modified Sodium Silicate on HIV-Protease Activity

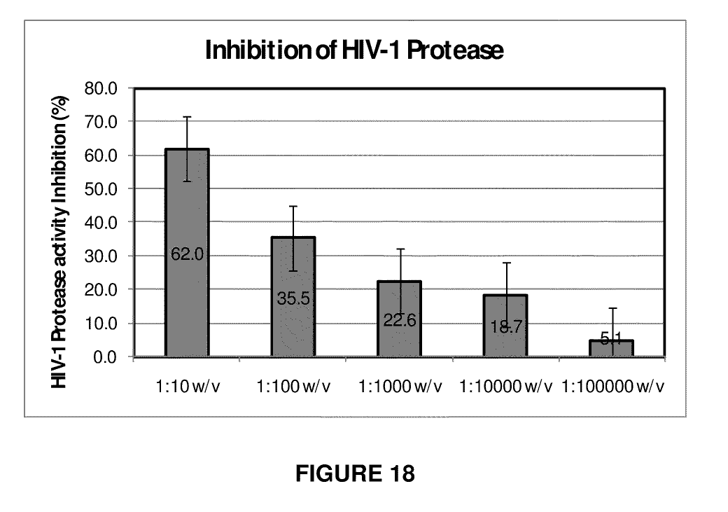

As described above, viral DNA, which is synthesized from reverse transcription, is transcribed and translated in the host cell to propagate infection. The viral DNA that is integrated into the host DNA is transcribed and translated into a single polypeptide chain. Different peptides needed for the viral assembly are generated by the post-translational modification of this single polypeptide chain. An important enzyme involved in this processing step is called the viral protease which cleaves the long chain into its individual enzyme components, which then facilitate the production of new viruses. In the absence of this viral protease activity, the viral assembly becomes improbable and therefore is an important target for controlling several types of retroviral infections. Diluted modified sodium silicate was diluted in DDI water 1:10; 1:100; 1:1000, 1:10000 and 1:100000s times and the effect of modified sodium silicate on HIV-1 protease activity using fluorescence measurements was measured. The modified sodium silicate was able to inhibit the protease activity in a linear dose dependent manner. At the highest concentration, (1:10) the compound was able to decrease the activity of the protease by 62% (FIG. 18). Further dilution caused 36%, 22% and 19% decrease in the protease activity. At the lowest concentration of the compound i.e. 1:100000, the inhibition activity was noted to be 5% (FIG. 18).

### Example 15

### Evaluation of Modified Sodium silicate on Anti-retroviral Activity

Treatment with this compound also resulted in an increased antioxidant response as seen from fold increases in activities of antioxidant enzymes SOD and CAT. The levels of important cellular antioxidant molecule GSH was also found to increase in a dose dependent manner. This was coupled with a decrease in MDA, an oxidative stress biomarker.

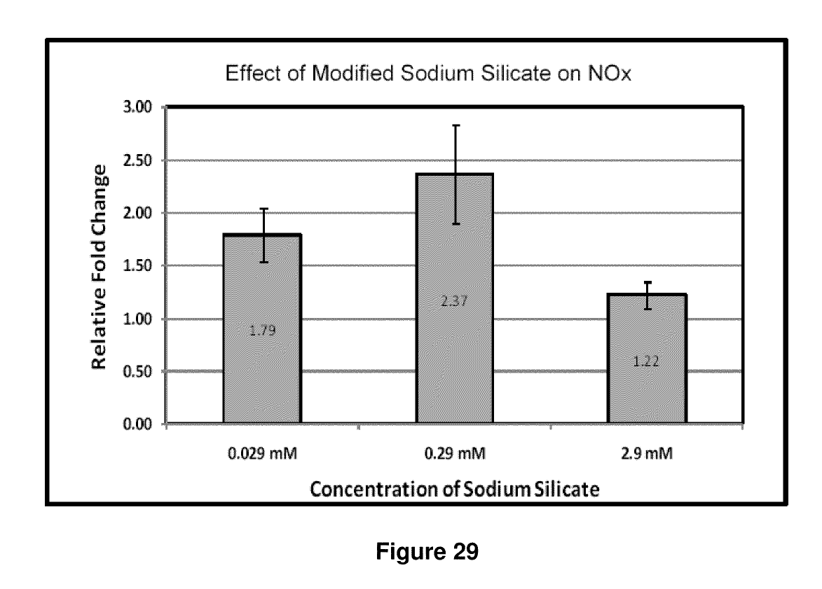

FIG. 29 illustrates the antioxidant and poptotic effects of modified sodium silicate.

Modified sodium silicate has the potential to decrease initial events in carcinogenesis by modulating redox mediated events, enhancing antioxidant response, promoting apoptosis and decreasing DONA mutations.

Additionally, modified sodium silicate inhibits viral infection, particularly retroviral infection, by inhibiting enzymes in viral assembly, changing the sugar composition in various and inhibiting enzymes responsible for transcribing RNA and DNA.

It is to be understood that the phraseology or terminology employed herein is for the purpose of description and not of limitation. The means and materials for carrying out disclosed functions may take a variety of alternative forms without departing from the invention. Thus, the expressions “means to . . . ” and “means for . . . ” as may be found the specification above, and/or in the claims below, followed by a functional statement, are intended to define and cover whatever structural, physical, chemical, or electrical element or structures which may now or in the future exist for carrying out the recited function, whether or not precisely equivalent to the embodiment or embodiments disclosed in the specification above, and it is intended that such expressions be given their broadest interpretation.

### BIBLIOGRAPHY

AIDS Epidemic Update—2004, UNAIDS/WHO (http://www.unaids.org)
Ames, B., McCann, J., and Yamasaki, E. Methods for Detecting Carcinogens and Mutagens with the Salmonella/Mammalian Microsome Mutagenicity Test. Mutation Research. 31: 347-364, 1975. 1975.
Bedoya et al., 2001 L. M. Bedoya, S. Sanchez-Palomino, M. J. Abad, P. Bermejo and J. Alcami, Anti-HIV activity of medicinal plant extracts, Journal of Ethnopharmcology 77 (2001), pp. 113-116.
Burke et al., 1995 T. R. Burke Jr., M. R. Fesen, A. Mazumder, J. Wang, A. M. Carothers, D. Grunberger, J. Driscoll, K. Kohn and Y. Pommier, Hydroxylated aromatic inhibitors of HIV-1 integrase, Journal of Medical Chemistry 38 (1995), pp. 4171-4178.
Coffin, J. M., HIV population dynamics in vivo: Implications for genetic variation, pathogenesis, and therapy. Science, 1995, 267, 483-488.
Cos et al., 2004 P. Cos, L. Maes, D. Vanden Berghe, N. Hermans, L. Pieters and A. Vlietinck, Plant substances as anti-HIV agents selected according to their putative mechanism of action, Journal of Natural Products 67 (2004), pp. 284-293.
Cos, P., Mees, L., Berghe, D. V., Hermans, N., Pieters, L. and Vlietinck, A., Plant substances as anti-HIV agents selected according to their putative mechanism of action. J. Nat. Prod., 2004, 67, 284-293.
De Clercq, 2000 E. De Clercq, Current lead natural products for the chemotherapy of human immunodeficiency virus (HIV) infection, Medicinal Research Review 20 (2000).
De Clercq, E., Antiviral therapy for human immunodeficiency virus infections. Clin. Microbiol. Rev., 1995, 8, 200-239.
Grant et al., 2002 R. M. Grant, F. M. Hecht, M. Warmerdam, L. Liu, T. Liegler, C. J. Petropoulos, N. S. Hellmann, M. Chesney, M. P. Busch and J. O. KALKA-V6™ n, Time trends in primary HIV-1 drug resistance among recently infected persons, Journal of the American Medical Association 288 (2002), pp. 181-188.
Half, E., Tang, X. M., Gwyn, K., ALKA-V6™ in, A., Wathen, K. and Sinicrope, F. A. Cyclooxygenase-2 expression in human breast cancers and adjacent ductal carcinoma in situ. Cancer Res. 62(6), 1676-81. 2002.
Hiramoto, K., Yasuhara, Y., Sako, K., Aoki, K. and Kikugawa, K. Suppression of free radical-induced DNA strand breaks by linoleic acid and low density lipoprotein in vitro. Biol Pharm Bull. 26(8):1129-34. 2003.
Jung, M., Lee, S., Kim, H. and Kim, H., Recent studies on natural products as anti-HIV agents. Curr. Med. Chem., 2000, 7, 649-661.
Laemmli, U. K. Cleavage of structural proteins during the assembly of the head of bacteriophage T4. Nature 227, 680-685, 1970.
Matthee, G., Wright, A. D. and Konig, G. M., HIV reverse transcriptase inhibitors of natural origin. Planta Med., 1999, 65, 493-506.
Nordling, M. M., Nygren, J., Bergman, J., Sundberg, K. and Rafter, J. J.

Toxicological characterization of a novel in vivo benzo[a]pyrene metabolite, 7-oxo-benz[d]anthracene-3,4-dicarboxylic acid anhydride. Chem Res Toxicol. 15(10), 1274-80. 2002.
Seeram N P, Adams L S, Hardy M L, Heber D. Total cranberry extract versus its phytochemical constituents: antiproliferative and synergistic effects against human tumor cell lines. Agric Food Chem. 5; 52(9):2512-7. 2004
Towbin, H., Staehelin, T. and Gordon, J. Electrophoretic transfer of proteins from polyacrylamide gels to nitrocellulose sheets, procedure and some applications. Proc Natl Acad Sci USA 76, 4350-4354, 1979
Vattem, D. A., Jang, H-D., Levin, R. E. and Shetty, K. “Synergism of cranberry phenolics with ellagic acid and rosmarinic acid for antimutagenic and DNA-protection functions” Journal of Food Biochemistry (Submitted) 2004.
Yamauchi, N., Takezawa, T., Kizaki, K., Herath, C. B. and Hashizume. Proliferative potential of endometrial stromal cells, and endometrial and placental expression of cyclin in the bovine. J Reprod Dev. 49(6), 553-60. 2003.
Yang, S. S., Cragg, G. M., Newman, D. J. and Bader, J. P., Natural product-based anti-HIV drug discovery and development facilitated by the NCI developmental therapeutics program. J. Nat. Prod., 2001, 64, 265-277.

What is claimed is:

1. A modified sodium silicate composition comprising the empirical formula of Na_{8.2}Si_{4.4}H_{9.7}^{O}_{17.6}.

2. The modified sodium silicate composition of claim 2, which comprises one or more ionizable compounds in equilibrium with the each other.

3. The modified sodium silicate composition of claim 2, which comprises a mixture of:
trimeric sodium silicate (Na₂SiO₃)₃ and sodium silicate pentahydrate (Na₂SiO₃).5H₂O.

4. The modified sodium silicate composition of claim 3, wherein the sodium silicate pentahydrate (Na₂SiO₃).5H₂O exists in equilibrium in two structural forms of the following general formula:

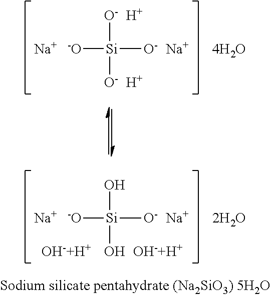

5. A pharmaceutical composition comprising the modified sodium silicate composition of claim 1, and optionally at least one pharmaceutically acceptable excipient or carrier.

6. The pharmaceutical composition of claim 5, further comprising one or more anti-cancer agents.

7. A method for treating cancer in a subject in need thereof, said method comprising causing one or more anti-cancer effects selected from the group consisting of preventing attachment of cancer cells; reducing harmful mutations in cellular DNA; inducing apoptosis; and stimulating anti-oxidant enzymes.

8. The method of claim 7, comprising causing the one or more anti-cancer effects by administering to the subject an effective amount of the silicon-based alkaline composition of claim 1.

9. The method according to claim 8, wherein the cancer is colon cancer.

10. The method of claim 8, wherein the modified sodium silicate composition is administered at a dose between 0.01 to 100 mg/kg body weight.

11. The method of claim 8, wherein the modified sodium silicate composition is administered at a dose between 0.1 to 100 mg/kg body weight.

12. The method of claim 8, wherein the modified sodium silicate composition is administered at a dose between 1 to 50 mg/kg body weight.

13. The method of claim 8, wherein the modified sodium silicate composition is administered one or more times and is optionally administered in combination with one or more anti-cancer agents.

14. The method of claim 8, wherein the method of administration is selected from the group consisting of parenteral, oral, aerosol, transdermal, parenteral, subcutaneous, intravenous, and combinations thereof.

15. A method for treating viral infection in a subject in need thereof, said method comprising causing one or more anti-viral effects selected from the group consisting of increasing nitric oxide dependent anti-viral effects; inhibiting enzymes involved in viral assembly; causing changes in viral carbohydrate composition; and inhibiting viral enzymes responsible for transcribing RNA to DNA.

16. The method of claim 15, comprising causing the one or more anti-viral effects by administering to the subject an effective amount of the modified sodium silicate composition of claim 1.

17. The method according to claim 16, wherein the viral infection is a retroviral infection.

18. The method according to claim 17, wherein the retroviral infection is caused by human immunodeficiency virus (HIV).

19. The method of claim 16, wherein the modified sodium silicate composition is administered at a dose between 0.01 to 100 mg/kg body weight.

20. The method of claim 16, wherein the modified sodium silicate composition is administered at a dose between 0.1 to 100 mg/kg body weight.

21. The method of claim 16, wherein the modified sodium silicate composition is administered at a dose between 1 to 50 mg/kg body weight.

22. The method of claim 16, wherein the modified sodium silicate composition is administered one or more times and is optionally administered in combination with one or more anti-viral agents.

23. The method of claim 16, wherein the method of administration is selected from the group consisting of parenteral, oral, aerosol, parenteral, subcutaneous, intravenous, and combinations thereof.
